SEQUENCE LISTING (12 sequences)

SEQ ID NO: 1 - DNA, 734 bp, Artificial Sequence
  Synthetic construct
atgagatgggccaacttccatctggaaaactcaggctggcaaaagatcaacaacttcagcgctgacatcaaggtaactgtgcaaaaaaccatagcgttcaggtagatacgatggcttcagggattttatcccattgtaacagagacaaaaatgataggatatcaccaggccctgttgtctttccctaggagctaatttccttaagtatattctgattttaaaaggttaaatgctctcttgccaaccatatgtgtttctcgtttaagcttttctgtatttcaacttttgatttaacatacaattaacatagtagatgttgtttccataggttgctactcatcatgctttgtaatgccataatgtgttcattttctctttccctatgcttcctggatttctgctcttctatttcatgcttgtttatctgtcaagcttattgacttcagtaacttcagtgaaggtacagaaatacttaatagctttattgggttgatggcatcgataaattgtcaagtcctaacaatcacgtgcaaagacaaaaagaaagatctctcaaataagaagacagagaagaatgccttatattatcacaaaagctgctcccaatgtcctcatcaccaagggacaataagatgcagctactctttggcagtgtgatcacactattatgttcaattcttttgtaggattccaaagcctatttccatctgctcaatcaaatcgcaccgaaggga

SEQ ID NO: 2 - DNA, 120 bp, Artificial Sequence
  Synthetic construct
atgagatgggccaacttccatctggaaaactcaggctggcaaaagatcaacaacttcagcgctgacatcaaggattccaaagcctatttccatctgctcaatcaaatcgcaccgaaggga

SEQ ID NO: 3 - DNA, 148 bp, Artificial Sequence
  Synthetic construct
atgagatgggccaacttccatctggaaaactcaggctggcaaaagatcaacaacttcagcgctgacatcaagcttattgacttcagtaacttcagtgaaggattccaaagcctatttccatctgctcaatcaaatcgcaccgaaggga

SEQ ID NO: 4 - DNA, 916 bp, Artificial Sequence
  Synthetic construct
atgatcaacttctatgcaggggcaaaccagagcatgaatgtcacttgtgttggcaaggtgagtgtgggggccctccttacctgcccacctggttagacttcctggtttctgagtgcttcacccatatctccctatctttttgtgctttcagaggccacagtcactagagggacaagggggtaagagtgggcgcctatgcagttttagctctaagaggctcttagccctattgcttctctctaggataaagagagcctgctgtcctggagatagacctatcccttcctgcaccaaagctctgacctctggttccttccctgtcaactttttcttacatctcagttgtctgggtttcttccactctcccatcatgccttgtttctcagttccctcagtctgctagctactgctcagttagcaccctttgctacaactagttgtccttggaaccctgcagccaactctgtcctctctagaaactctcctccttcccactgagccttgactgtttatctgttctttcttggctctgctccagagactgattcccaaggacggggtaagaacttggggattgatggtggagttagaaggccctcaccgtgttgtcagcacccttagaagacctagtctgatgggagataggccacccctatctgcagacatgcagataggaacatgtgtgcatgcgcacacacaaatgcacacacagctacctgagcagatgcacagctcaaagaaaacaagtttgacagggataatttgggatgaaggaggtacagaaggaagtcttgtgagcgcttccaggtgcctgctgttcctaacatcctctccccttaaccttcctgcacccccacagagagagaagatgctgagaaccttggccactttgtcatgttccctgctaatggcagca

SEQ ID NO: 5 - DNA, 114 bp, Artificial Sequence
  Synthetic construct
atgatcaacttctatgcaggggcaaaccagagcatgaatgtcacttgtgttggcaagagagagaagatgctgagaaccttggccactttgtcatgttccctgctaatggcagca

SEQ ID NO: 6 - DNA, 142 bp, Artificial Sequence
  Synthetic construct
atgatcaacttctatgcaggggcaaaccagagcatgaatgtcacttgtgttggcaagaggccacagtcactagagggacaaggggagagagaagatgctgagaaccttggccactttgtcatgttccctgctaatggcagca

SEQ ID NO: 7 - DNA, 990 bp, Artificial Sequence
  Synthetic construct
atgtgcaagaaaagaaaaggtcaagcatctggcaattgtgagtgagggccggggtcagacaagagcgttaccctgacctttcccagtggggaggagatagctaaactgaagttcttctctttaatatacagcatgccaactcgggtaaggctgtgctgcaggacccaaagtaggctgtcgtaagctgcactgtgttggttatgcagtggctggactcttctgctggtgtacataaaaccatgggggaaatggttctaaaattgacagtctcaggaatcctcaccaaagggatgaggaaaagtgtagtatttgtactgtttggttagttgtcttgtatgtgttaaacctgactttgtaccagaagtactgtgtgtttattgaagttaaaacttatccttttaagattaatctgtttactggaagcataaatgttttaaaactgtggacatgaaaatagttccttaagttgagcttagaaaattcatgctatgagttaacttggtgatgtgtgttctcagtgtagcagacctttgtatgcgctgcacactaagctttggctgttatcacggtcatccccctgctgctcactgaccactgtgactttgtcccacataggacctagctagccaaggagccttcgtattgtacttgtttgtaggagctttcagtacgcaagagcatctctttcattatggaacttgcttatatctagcattctaagcggtagctattcccccgagtgtccaagtttggttgtgactgactgctaaggcgatagtataaccataaagctggttttgctaatgtctaactcactgccttgtaaagttttgaaattttcttgattataaaaatttatggaaaattggggcttggttttcacaatgcatgtaataatagacatatctgtgggttttgtttgcctcttttattttgttagtttcagccgactttttagctcttcaagtaatgcaacaaagaagcctgaac

SEQ ID NO: 8 - DNA, 87 bp, Artificial Sequence
  Synthetic construct
atgtgcaagaaaagaaaaggtcaagcatctggcaatttttcagccgactttttagctcttcaagtaatgcaacaaagaagcctgaac

SEQ ID NO: 9 - DNA, 100 bp, Artificial Sequence
  Synthetic construct
atgtgcaagaaaagaaaaggtcaagcatctggcaattcatgccaactcggtttcagccgactttttagctcttcaagtaatgcaacaaagaagcctgaac

SEQ ID NO: 10 - DNA, 1599 bp, Artificial Sequence
  Synthetic construct
atggatcaaaaacgctcaggagctggcttgtggagtgtgtctcttaaatgtggactccaggagccgggtaagtcctggccttcctccttcaagggccattgtttatccctaacatgatttaaggggcagccacatgtagcccccttcacaagtgctgtgaccagaggtggggatgctgaagtctagaaaggtctatagaagcagatctgttttttccacagggcagtggggaactgctgagggattaaacagagtcctccctccctgctgagcattggattcacccgggacaggggtgggtggagccagaaggccaggccctgtggacttacctggaggcaagagccgttccaggggtttgattagctttttcttttgaccttatggtttgaacatttcagaaagaagagacgcctgcagaaggaacagcctaaaaaagtatgagccagctacatgttactttagtgtctccatctcagtcttgtgtgtgagaggccaacagataattctgttgttgtcctggactgtaccagttgtgaatcctctgctgactcactcgtccccataacgtgtgatcaaatcaactaatgcgtctctgtggggccactgccgtcctgtttgcgtgcttgtagctgctaatcagcttctgctgttgtccgtatctttcctcaccacgaatcagcaccttctggaagactagaagttgtagtgacagagaatgagatggcagagcgtcaggatttagtggatgagagacaaaaaaagaaaaaaatcaaatcatttgggttctaggattttaactgtagtgtttaagaaaacaaactgcaggctgttacctgctgtgggtagctgaaggagggtctcatatgtccactcagttgtcagcccccaatggcagtggaccagtgattctcaaccttcctgatgctgctgcgaccctttaatacagttcttcatggtgtgttgacccccaagcataaaattattttgtgggtaccgtaatttcgctgctattagaagtcgtaatgtaaatacctggcatgggtcctgactcacaggttgagaactgctgcatgagactttaagagttaatatcaattgatttctaactcttttaaatgttttcagtcctgatagaactgtaattagccacagacctaaggggccacatgttacagcagtggcagcctccagggagggtggctattatagtgagggaaccaattacccacaggccctggctattttgattcccgtgccctgcccctttttctctcctgtaacctctgcctgccttttctgtgaagattggcccttttgctgaaactaggttggatggtactactgactttatcctttaattagcagtaaaaatgaataaaactttcctacttggatcctgtagccacctgctcggcttttgtctgtgtgtgccatttgggcctcagccttggtgacacctggggagtgtccgtagtggttgctgacaagctctcagatgtagaaagaccctgacttgcccccatctctctatttctggcaggcattcaactcggagacagacagcttcaagctggcctacggaggacaccagtacca

SEQ ID NO: 11 - DNA, 123 bp, Artificial Sequence
  Synthetic construct
atggatcaaaaacgctcaggagctggcttgtggagtgtgtctcttaaatgtggactccaggagccgggcattcaactcggagacagacagcttcaagctggcctacggaggacaccagtacca

SEQ ID NO: 12 - DNA, 160 bp, Artificial Sequence
  Synthetic construct
atggatcaaaaacgctcaggagctggcttgtggagtgtgtctcttaaatgtggactccaggagccggaaagaagagacgcctgcagaaggaacagcctaaaaaagcattcaactcggagacagacagcttcaagctggcctacggaggacaccagtacca

(19) United States
(12) Patent Application Publication
(10) Pub. No.: US 2024/0108756 A1
(43) Pub. Date: Apr. 4, 2024

(54) COMPOSITIONS AND METHODS FOR USING ALTERNATIVE SPLICING TO CONTROL SPECIFICITY OF GENE THERAPY

(71) Applicant: The Johns Hopkins University, Baltimore, MD (US)

(72) Inventors: Jonathan P. Ling, Baltimore, MD (US); Seth Blackshaw, Baltimore, MD (US)

(21) Appl. No.: 17/768,591

(22) PCT Filed: Oct. 16, 2020
(86) PCT No.: PCT/US2020/056156

§ 371 Date: Apr. 13, 2022

Related U.S. Application Data
(60) Provisional application No. 62/916,396, filed on Oct. 17, 2019.

(51) Int. Cl. A61K 48/00; C12N 15/86

(52) U.S. Cl. CPC A61K 48/0058; C12N 15/86; C12N 2740/15042; C12N 2740/15045; C12N 2740/15071; C12N 2750/14143; C12N 2750/14145; C12N 2750/14171; C12N 2830/42; C12N 2840/445

(57) ABSTRACT

Disclosed herein are compositions and methods that can be used to express a nucleotide sequence in a specific cell type. The compositions can comprise nucleic acid constructs comprising a start codon; and an intron cassette. The intron cassette can comprise a cell specific exon sequence, a splice donor site, a branch site, and an acceptor site. The cell specific exon sequence is out of frame with the start codon and comprises one or more frameshift mutations. The compositions can be used to treat human diseases.

### CROSS REFERENCE TO RELATED APPLICATIONS

This application claims the benefit of priority of U.S. Provisional Application No. 62/916,396, filed Oct. 17, 2019. The content of this earlier filed application is hereby incorporated by reference herein in its entirety.

### INCORPORATION OF THE SEQUENCE LISTING

The present application contains a sequence listing that is submitted via EFS-Web concurrent with the filing of this application, containing the file name “36406_0015P1_SL.txt” which is 12,288 bytes in size, created on Oct. 6, 2020, and is herein incorporated by reference in its entirety.

### BACKGROUND

Viral-based gene therapy holds tremendous promise for the treatment of human diseases. However, an important concern when designing gene therapy vectors is to ensure that genes are only delivered to the intended cell type, since off-target delivery can lead to side effects and toxicity. Strategies to overcome this issue include using different viral coat proteins or custom minimal promoters. However, these methods have significant limitations.

### SUMMARY

Disclosed herein are a nucleic acid constructs comprising: a) a start codon; and b) an intron cassette, wherein the intron cassette comprises a cell specific exon sequence, a splice donor site, a branch site, and an acceptor site, wherein the cell specific exon sequence is out of frame with the start codon and comprises one or more frameshift mutations.

Disclosed herein are nucleic acid constructs comprising: a) a first intron sequence comprising a constitutive splice donor site, a branch site, and an alternative splice acceptor site; b) a cell specific exon sequence; and c) a second intron sequence comprising an alternative splice donor site, a branch site, and an constitutive splice acceptor site.

Disclosed herein are nucleic acid constructs comprising from 5′ to 3′: a first intron sequence comprising a constitutive splice donor site, a branch site, and an alternative splice acceptor site; a cell specific exon sequence; and a second intron sequence comprising an alternative splice donor site, a branch site, and an constitutive splice acceptor site.

### BRIEF DESCRIPTION OF THE DRAWINGS

FIG. 1 shows the identification of thousands of alternative exons enriched across the nervous system using ASCOT. Mouse RNA-Seq datasets were manually curated from the Sequence Read Archive (SRA), covering a broad range of cell types and organs. Cell type datasets were generated from various independent labs using FACS or affinity isolation. To test a splicing detection algorithm, alternative exons were identified that were differentially spliced between neuronal cell types and other cell types in the body. Exons could be generally clustered by their inclusion or exclusion in various organs and cell types such as rods, neurons, muscles, pancreas, or non-neuronal tissues. Each row is an individual exon, and exon utilization is measured by a percent spliced in (PSI) ratio as indicated by gradient legend.

FIG. 2 shows an example of a highly cell type-specific exon. An alternative exon in Sptan1 is found in cochlear hair cells, despite ubiquitous expression across other organs and cell types. PSI=percent spliced-In, NAUC=normalized area under the curve, equivalent to gene expression.

FIG. 3 shows a diagram of a bichromatic fluorescent reporter. In this construct, a 5′ upstream exon contains the start codon (ATG). Depending on whether an alternative exon is spliced into the mRNA, two coding sequences can be generated: one that is in-frame with mCherry and another that is in-frame with GFP and reads through the mCherry sequence. The mCherry sequence is modified to remove any stop codons that would terminate the GFP reading frame. Importantly, alternative splicing is independently regulated and thus any promoter (strong or weak, selective or ubiquitous) can be used to drive gene expression. An intersectional approach can be used whereby selective promoters and cell type-specific alternative exons can target specific cell types.

FIGS. 4A-Q show the validation of plasmid-based SLED constructs across mouse, rat and human. GFP fluorescence is detected only in the relevantly targeted cell types for Muscle-SLED (human, A-D), Neuron-SLED (rat, E-H), Glutamatergic-SLED (rat, I-L), and Rod-SLED (mouse, M-Q). (A-D, human fibroblasts and myotube cultures) GFP is not detected in fibroblasts (arrows, A and B) but is detected in myotubes (asterisks, C and D). (E-H, rat hippocampal cultures) GFP is detected in hippocampal neurons (asterisks) but not detected in glia (arrows), as indicated by NeuN staining (H). (I-L, rat hippocampal cultures) Excitatory neurons express GFP (asterisks), but somatostatin-positive interneurons do not, as indicated by somatostatin immunostaining (arrows) (L). (M-Q, mouse retina explants) Photoreceptors in the outer nuclear layer (asterisk in Q) express NLS-GFP, while other cell types in the inner nuclear layer (arrow in Q) do not, as indicated by recoverin (N) and DAPI (P). Scale bar=100 μm.

FIGS. 5A-L show AAV delivery of muscle-SLED and neuron-SLED retains cell type specificity. FIGS. 5A-D show E17 rat cortical cultures were infected with AAV9 muscle-SLED and AAV9 neuron-SLED. FIGS. 5E-H show in vivo delivery of AAV9 neuron-SLED into 2 month old mouse hippocampus via parenchymal injection. FIG. 5G show NeuN stain, with acellular punctate mouse-on-mouse autoreactivity observed. FIG. 5H show a DAPI counterstain. FIG. 5I-L show in vivo delivery of AAV9 Muscle-SLED into 2 month old mouse quadriceps muscle. Wheat germ agglutinin counterstain indicates muscle fibers (FIG. 5K); FIG. 5L shows the DAPI counterstain. Scale bar=100 μm.

FIG. 6 shows that MULTI-Seq enables cost-effective gene expression analysis. MULTI-Seq was used to multiplex 8 E18 retinal explant samples in a single 10× sequencing run. Cells are evenly distributed among the observed cell types (RPCs=retinal progenitor cells, AC=amacrine cells, PR=photoreceptors, RGCs=retinal ganglion cells).

FIG. 7 shows the hierarchical classification of nervous system cell types; a simplified schematic describing taxonomic levels of cell identity delineated by gene expression and alternative splicing. Cell types already successfully targeted using SLED constructs are indicated by checkmarks.

FIG. 8 shows intron length distribution of alternative exons. Ranking intron sequences by length, cell type-specific exons exhibit a similar distribution compared to alternative exons. It is estimated that ˜30-37% of exons are suitable for AAV delivery (<3 kb) without needing to trim the intron sequence. Alternatively, ˜60% of exons are suitable for lentiviral or nanoparticle delivery (<6 kb).

FIGS. 9A-K show examples of cell type-specific alternative exons to drive cell type-specific gene expression. FIG. 9A is a schematic of neuron-specific expression plasmid. FIG. 9B show mCherry expression, FIG. 9C shows GFP expression, FIG. 9D shows neuronal marker expression, and FIG. 9E shows mCherry and GFP overlay. FIG. 9F is a schematic of photoreceptor-specific expression plasmid. FIG. 9G shows GFP, FIG. 9H shows photoreceptor marker, FIG. 9I shows mCherry, FIG. 9J shows nuclear stain, and FIG. 9K shows mCherry and GFP overlay.

FIGS. 10A-F shows the results of using alternative splicing to induce photoreceptor-specific gene expression in the retina. Electroporated retinas demonstrate a clear difference in fluorescence between the photoreceptor layer and non-photoreceptor layer, as only photoreceptors are green (FIG. 10A), while non-photoreceptor cells are red (FIG. 10B). Red-green overlay (FIG. 10C), nuclei stain (FIG. 10D), triple overlay (FIG. 10E), and diagram of retina cell layers (FIG. 10F) are shown.

FIG. 11 is a schematic of a nucleic acid construct useful for controlling gene expression with alternative splicing in specific cell types.

FIGS. 12A-C show the results of using alternative splicing to induce gene expression specific to cells with oncogenic SF3B1 mutations. FIG. 12A shows RTPCR analysis across three uveal melanoma cell lines (92.1, OMM1, Mel202). FIG. 12B is a schematic of an SF3B1 mutation-specific expression plasmid. FIG. 12C shows differential mCherry and GFP expression across uveal melanoma cell lines.

FIGS. 13A-L show that alternative splicing can be used to rescue the rd2 mouse model. FIGS. 13A, D, G, and J show DAPI nuclear stains. FIGS. 13B, E, and H show immunohistochemical staining of Prph2. FIG. 13K shows GFP fluorescence. FIGS. 13C, F, I, and L show composite overlays. Scale bar=100 μm.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to the administering step.

As used herein, the term “comprising” can include the aspects “consisting of” and “consisting essentially of.”

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “expression vector” is herein to refer to vectors that are capable of directing the expression of genes to which they are operatively-linked. Common expression vectors of utility in recombinant DNA techniques are often in the form of plasmids. Recombinant expression vectors can comprise a nucleic acid as disclosed herein in a form suitable for expression of the acid in a host cell. In other words, the recombinant expression vectors can include one or more regulatory elements or promoters, which can be selected based on the host cells used for expression that is operatively linked to the nucleic acid sequence to be expressed.

The term “sequence of interest” or “gene of interest” can mean a nucleic acid sequence (e.g., a therapeutic gene), that is partly or entirely heterologous, i.e., foreign, to a cell into which it is introduced.

The term “sequence of interest” or “gene of interest” can also mean a nucleic acid sequence, that is partly or entirely homologous to an endogenous gene of the cell into which it is introduced, but which is designed to be inserted into the genome of the cell in such a way as to alter the genome (e.g., it is inserted at a location which differs from that of the natural gene or its insertion results in “a knockout”). For example, a sequence of interest can be cDNA, DNA, or mRNA.

The term “sequence of interest” or “gene of interest” can also mean a nucleic acid sequence that is partly or entirely complementary to an endogenous gene of the cell into which it is introduced.

A “sequence of interest” or “gene of interest” can also include one or more transcriptional regulatory sequences and any other nucleic acid, such as introns, that may be necessary for optimal expression of a selected nucleic acid. A “protein of interest” means a peptide or polypeptide sequence (e.g., a therapeutic protein), that is expressed from a sequence of interest or gene of interest.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

The terms “alter” or “modulate” can be used interchangeable herein referring, for example, to the expression of a nucleotide sequence in a cell means that the level of expression of the nucleotide sequence in a cell after applying a method as described herein is different from its expression in the cell before applying the method.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, malady, disorder, sickness, illness, complaint, or affection.

As used herein, the terms “promoter,” “promoter element,” or “promoter sequence” are equivalents and as used herein, refers to a DNA sequence which when operatively linked to a nucleotide sequence of interest is capable of controlling the transcription of the nucleotide sequence of interest into mRNA. A promoter is typically, though not necessarily, located 5′ (i.e., upstream) of a nucleotide sequence of interest (e.g., proximal to the transcriptional start site of a structural gene) whose transcription into mRNA it controls, and provides a site for specific binding by RNA polymerase and other transcription factors for initiation of transcription.

Suitable promoters can be derived from genes of the host cells where expression should occur or from pathogens for this host cells (e.g., tissue promoters or pathogens like viruses). If a promoter is an inducible promoter, then the rate of transcription increases in response to an inducing agent. In contrast, the rate of transcription is not regulated by an inducing agent if the promoter is a constitutive promoter. Also, the promoter may be regulated in a tissue-specific or tissue preferred manner such that it is only active in transcribing the associated coding region in a specific tissue type(s) such as leaves, roots or meristem. The term “tissue specific” as it applies to a promoter refers to a promoter that is capable of directing selective expression of a nucleotide sequence or gene of interest to a specific type of tissue in the relative absence of expression of the same nucleotide sequence or gene of interest in a different type of tissue.

Although the foregoing invention has been described in some detail by way of illustration and example for purposes of clarity of understanding, certain changes and modifications may be practiced within the scope of the appended claims.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Disclosed herein is a technology that was developed that uses alternative splicing to precisely restrict gene expression to certain cell types. By coupling cell type-specific splicing events to a desired translational reading frame, it can be ensured that a virus will preferably express proteins in the target cell type. Importantly, this technology functions independently from other methods of controlling gene therapy and can therefore be used in conjunction with other strategies. Furthermore, the genomic sequences used in this technology are compatible across species and will lead to a higher likelihood of successful translation from animal model to clinic.

The compositions and methods disclosed herein can be used for drug delivery using gene therapy. Nucleic acid constructs disclosed herein can be used to drive cell type-specific gene expression (independent of any other sequence in the plasmid). These nucleic acid constructs disclosed herein can be incorporated into constructs for viral-based gene therapy.

The mammalian nervous system is comprised of a diverse range of functionally distinct cell types. A detailed understanding of the cell types and connections that underlie cognition and behavior is still lacking. New tools are urgently needed to selectively target specific cell types and monitor or manipulate neuronal activity without having to rely on genetic manipulation. Were these tools to become available, it would allow detailed mechanistic investigation of neural circuitry in a broad range of mammalian species.

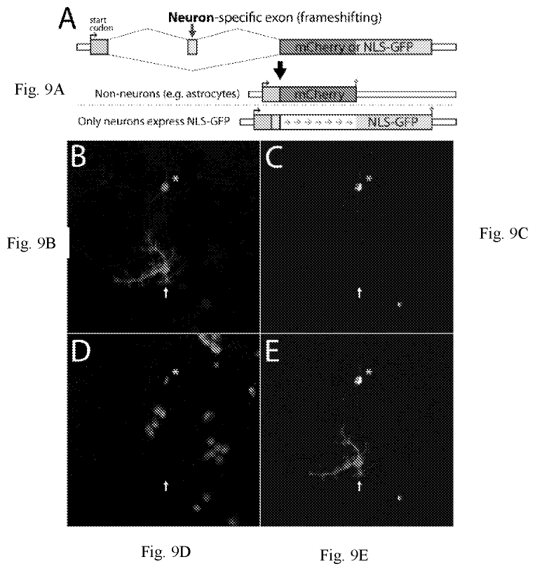

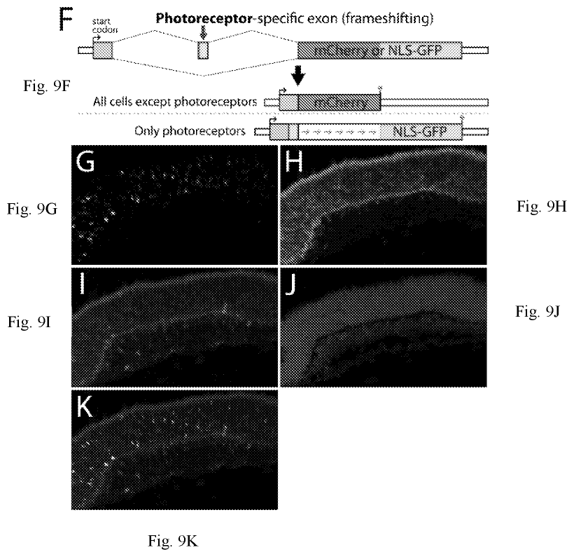

Disclosed herein are molecular tools that use alternative splicing events to drive cell type-specific gene expression. By coupling expression of reporter and effector genes to splicing of highly cell type-specific and evolutionarily conserved alternative exons, cell type-specific expression of these genes can be driven in wild-type animals from many different mammalian species. Using a recently developed database of alternative splicing across thousands of bulk and single cell RNA-Seq datasets from the public archive, intron and exon sequences were identified that were suitable for use in the splicing-linked expression design (SLED). As a proof of concept, SLED constructs were created that selectively express alternative exon-dependent GFP in photoreceptors, muscles, or neurons (see e.g., FIG. 9 and Example 5). The constructs used alternative exons that are conserved across mammals and, as expected, it was observed that cell type-specificity is retained across mouse, rat and human cells. Furthermore, specificity is also retained when these constructs are delivered using AAV or lentivirus.

There are likely to be thousands of cell types in the central and peripheral nervous systems. These form the basic building blocks of neural circuitry. Transgenic approaches have had great success in selectively labeling individual neuronal subtypes, which is important for rigorous analysis of neural circuitry. There are now many transgenic and knock-in lines available that target expression of fluorescent proteins or Cre recombinase to specific cell types (Gong S, et al. (2003) Nature 425(6961):917-925; and Taniguchi H, et al. (2011) A resource of Cre driver lines for genetic targeting of GABAergic neurons in cerebral cortex. Neuron 71(6):995-1013). Despite the vast amount of effort invested in generating these resources, there are nonetheless several important limitations in using transgenic approaches for neuronal subtype identification.

First, generating transgenic animals is expensive and laborious. The expense and logistics of maintaining these lines is daunting, and increasingly vulnerable to disruption by natural and man-made disasters (Fishell G (2013) Hurricane Sandy: After the deluge. Nature 496(7446):421-422; and Culliton B J (1989) Fire devastates Jackson lab. Science 244(4906):767-768). Second, transgenic animals are usable in genetically tractable organisms. Although technologies such as Cas9/CRISPR have made it feasible to somewhat broaden this pallet, and make it possible to generate transgenic models in species not widely used for genetic studies, such as marmosets and macaque monkeys (Niu Y, et al. (2014) Cell 156(4):836-843; and Kumita W, et al. (2019) Sci Rep 9(1):12719), the fact remains that brain circuitry in higher primates and humans is slow and expensive to study. Third, transgenic reagents are by their nature species-specific. Separate lines must be generated and maintained in order to investigate similar cell types in different model organisms. Finally, mapping neuronal circuitry requires the use of different labels when multiple neuronal subtypes are studied. Using conventional transgenic approaches, it becomes progressively harder and more expensive to analyze additional markers in a single experiment, effectively becoming impractical when more than three or four different transgenes must be combined. To bypass these obstacles, techniques are needed that allow control of gene expression to be directed to specific neuronal subtypes, but which does not require genetic modification of the target cells, and can be selectively applied in spatially restricted regions of the nervous system. New, more efficient approaches for generating viral vectors that drive cell type-specific expression, and which can be combined intersectionally with existing cis-regulatory element-based reagents to achieve higher levels of cell type-specific expression, are urgently needed.

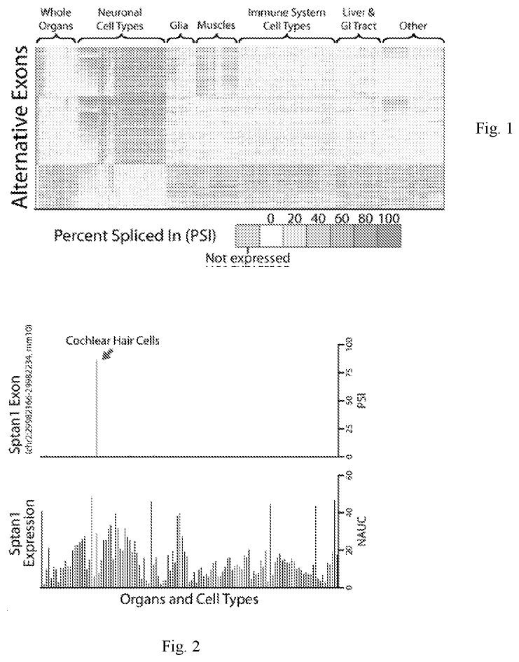

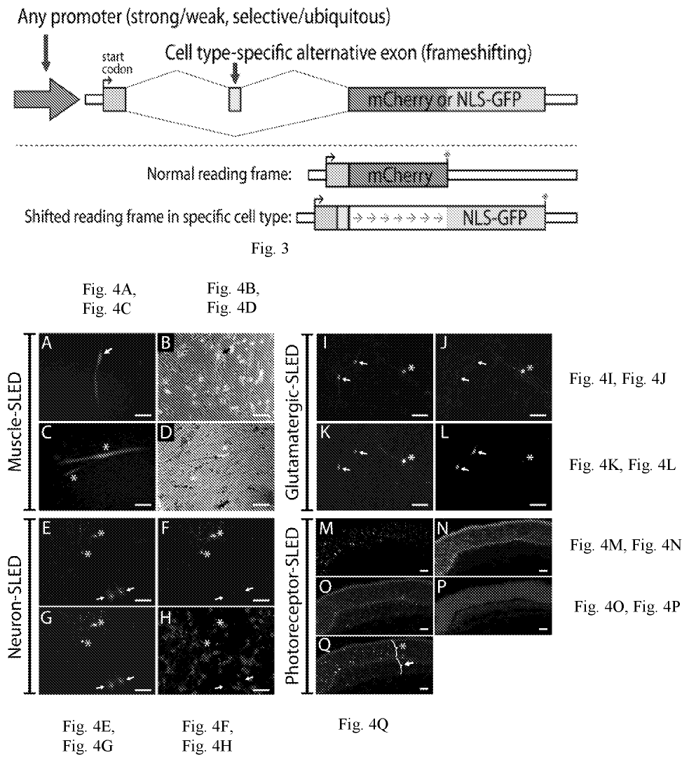

As described herein, an alternative approach has been developed. The scientific premise of this approach is that, by making use of cell type-specific patterns of alternative splicing, it will be possible to design viral vectors that show highly specific expression. Using ASCOT, a computational tool that allows rapid identification and annotation of cell type-specific alternative exons, public RNA-Seq datasets have been analyzed to comprehensively profile cell type-specific patterns of alternative splicing, in the process identifying many previously unannotated alternative exons (Ling J P, et al. (2018) ASCOT identifies key regulators of neuronal subtype-specific splicing. bioRxiv. doi:10.1101/501882). Many exons show varying levels of specificity to individual nervous system cell types (FIG. 1). Particularly in the case of primary sensory neurons—such as rod photoreceptors, olfactory sensory neurons and somatosensory neurons of the dorsal root ganglion (DRG)—some of these alternative exons are cell type-specific (FIG. 2). In other cases, exons may be shared with a subset of other neuronal or non-neuronal cell types (FIG. 1). This data has been used to carry out SLED, using both alternative and ubiquitous exons to drive expression of a two-color fluorescent reporter (FIG. 3). SLED has also been used to drive rod photoreceptor, muscle and excitatory neuron-specific reporter expression using plasmid and/or viral-based expression systems.

SLED-based viral reagents can also be developed that target a broad range of cell types in the peripheral and central nervous system. Using SLED alone, primary sensory and motor neurons, along with cortical excitatory and inhibitory neurons, astrocytes and oligodendrocytes can be targeted. By combining cell type-specific promoter elements and SLED, viral vectors can be generated that will target layer specific pyramidal neurons and specific subtypes of inhibitory cortical neurons. It can then be confirmed that these reagents express appropriately in a broad range of mammalian species, and that they can efficiently express constructs that allow monitoring and manipulation of activity levels in these specific cell types.

SLED has several innovative features that make it potentially transformative. First and foremost, by using evolutionarily conserved patterns of cell type-specific splicing to drive cell type-specific expression of reporter and effector constructs, it provides a scalable approach for selectively targeting expression of reporter and effector constructs to virtually any CNS cell type in wild type animals. This frees investigators from relying on the expensive and slow process of transgenesis to accomplish this goal. Second, intersectional combination of SLED with different promoter elements can generate constructs with higher or altered specificity, and differing levels of expression. Finally, SLED vectors generated in this study should be equally useful in rodents, carnivores, non-human primates and humans. This is not possible with any currently available approach, and is directly relevant to the understanding of the human brain—the ultimate goal of the BRAIN Initiative.

The ability to selectively target reporter and effector gene expression to specific cell types in the nervous system is currently very limited, and relies heavily on the use of cis-regulatory promoter and enhancer elements of highly cell type-specific genes to regulate expression of reporter and/or effector genes in either AAV or lentiviral constructs. Following a great deal of effort, a small number of useful constructs of this type are available. In the retina, AAV-based constructs using the L/M cone opsin (Ye G-J, et al. (2016) Hum Gene Ther 27(1):72-82.) and Rlbp1 (Pellissier L P, et al. (2014) Mol Ther Methods Clin Dev 1:14009) promoters respectively target rod photoreceptors and Muller glia with reasonably high specificity. Synapsin I (Glover C P J, et al. (2002) Mol Ther 5(5 Pt 1):509-516; and Kügler S, Kilic E, Bahr M (2003) Gene Therapy 10(4):337-347.) and CamKII promoters (Dittgen T, et al. (2004) Proc Natl Acad Sci USA 101(52):18206-18211) respectively drive pan-neuronal and excitatory neuron-specific expression in cortex. In a few cases, the use of promoters of highly cell type-specific neuropeptide, such as MCH, has achieved cell type-specific expression when used in viral vectors (van den Pol A N, et al. (2004) Neuron 42(4): 635-652).

In the majority of cases, however, evolutionarily-conserved cis-regulatory elements for cell type specific genes are not sufficient to drive appropriate expression when used in heterologous viral vectors (Nathanson J L (2009) Frontiers in Neural Circuits 3. doi:10.3389/neuro.04.019.2009). Even in cases where viral constructs of this sort do show cell type-specific expression in a given species, this expression is often not evolutionarily conserved, even when the sequence of the element itself is highly conserved (Ye G-J, et al. (2016) Hum Gene Ther 27(1):72-82.). Systematic efforts to overcome these problems have met with some success. Notably, vectors using ultraconserved enhancers of Dlx1/2 have been used to selectively target forebrain interneurons in a wide variety of species (Dimidschstein J, et al. (2016) Nat Neurosci 19(12):1743-1749), while minipromoters constructed of target sites for highly cell type-specific transcription factors have been used to generate AAV vectors that selectively target rods, cones, Muller glia, bipolar and amacrine cells in mouse, macaque and human retina (Jüttner J, et al. (2019) Nat Neurosci 22(8):1345-1356). More recently, integration of snRNA-Seq and ATAC-Seq data has guided the design of AAV vectors that selectively target cortical Sst-positive neurons (Hrvatin S, et al. (2019) PESCA: A scalable platform for the development of cell-type-specific viral drivers. bioRxiv:570895.).

Even in these cases, however, the success rate for constructs tested was low. Some have also been reported to show leaky expression in certain contexts (Dimidschstein J, et al. (2016) Nat Neurosci 19(12):1743-1749; and Wilson D E, et al. (2017) Neuron 93(5):1058-1065.e4.). Importantly, even when such vectors express appropriately, they are not useful where combinatorial patterns of gene expression rather than expression of a single gene define cell type identity, as is the case for most cell types in the nervous system (Zeng H, Sanes J R (2017) Nat Rev Neurosci 18(9):530-546). Although individual cis-regulatory elements cannot be readily combined in a simple Boolean manner, complementary approaches that can be combined with existing cell type-specific cis-regulatory elements may be able to drive highly selective patterns of cell type-specific expression.

### Compositions

Nucleic acid constructs. Disclosed herein are nucleic acid constructs. Any combination of the nucleic acid construct disclosed herein can be present in a single nucleic acid construct. Table 1 provides examples of intron cassette sequences as well as sequences that can be present in certain cells after splicing using the compositions and methods described herein.

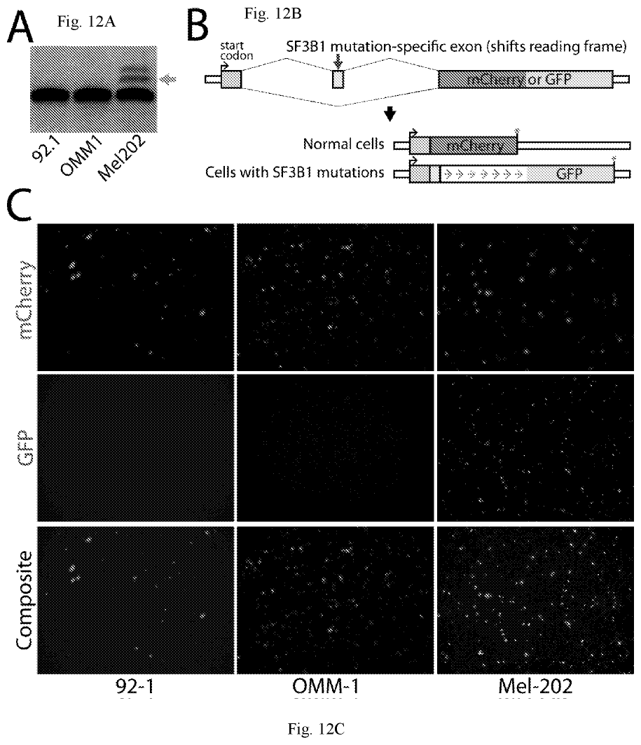

Described herein are nucleic acid constructs comprising: a start codon and an intron cassette. In some aspects, the intron cassette can comprise a cell specific exon sequence, one or more splice donor sites, one or more branch sites, and one or more acceptor sites. In some aspects, the cell specific exon sequence is out of frame with the start codon and comprises one or more frameshift mutations. In some aspects, the cell specific exon sequence 3′ of a first splice donor site branch site and splice acceptor site and 5′ of a second splice donor site branch site and splice acceptor site. An example of a disclosed construct is shown in FIG. 11.

Also described herein are nucleic acid constructs comprising: a) a first intron sequence comprising a constitutive splice donor site, a branch site, and an alternative splice acceptor site; b) a cell specific exon sequence; and c) a second intron sequence comprising an alternative splice donor site, a branch site, and an constitutive splice acceptor site. Further described herein are nucleic acid constructs comprising from 5′ to 3′: a first intron sequence comprising a constitutive splice donor site, a branch site, and an alternative splice acceptor site; a cell specific exon sequence; and a second intron sequence comprising an alternative splice donor site, a branch site, and an constitutive splice acceptor site. In some aspects, the nucleic acid constructs can further comprise a start codon. In some aspects, the start codon can be upstream from the first intron sequence. In some aspects, the cell specific exon sequence can be flanked by the first intron sequence and the second intron sequence. In some aspects, the nucleic acid constructs can further comprise a Kozak sequence. In some aspects, the Kozak sequence can be upstream of the first intron sequence. In some aspects, the Kozak sequence is upstream of the first intron sequence and is out of frame with the cell specific exon sequence. In some aspects, the nucleic acid constructs can further comprise a gene of interest. In some aspects, the gene of interest is downstream of the second intron sequence.

Start codons. In some aspects, a start codon can be upstream from the intron cassette. In some aspects, the start codon can be ATG. In some aspects, the nucleic acid constructs disclosed herein can further comprise a 5′ untranslated region (5′UTR). In some aspects, the start codon can be preceded by a 5′UTR. In some aspects, the 5′UTR can be positioned between the promoter and the start codon. In some aspects, the nucleic acid constructs disclosed herein can further comprise a Kozak sequence. In some aspects, the 5′UTR can be positioned between the promoter and the Kozak sequence. In some aspects, the start codon can be preceded by a Kozak sequence. In some aspects, the start codon can be directly preceded by a Kozak sequence. In some aspects, the Kozak sequence can be upstream of the intron cassette. In some aspects, the Kozak sequence can be upstream of the intron cassette and is out of frame with the cell specific exon sequence. In some aspects, the start codon can be preceded by both a 5′UTR and a Kozak sequence (see, for example, FIG. 11).

In some aspects, the nucleic acid constructs disclosed herein can further comprise a polyadenylation signal. In some aspects, the nucleic acid constructs disclosed herein can further comprise a 3′ untranslated region (3′UTR). In some aspects, the polyadenylation signal can be preceded by a 3′UTR. In some aspects, the 3′UTR can be positioned between the gene of interest and the polyadenylation signal (see, for example, FIG. 11).

The term “codon” denotes an oligonucleotide consisting of three nucleotides that encodes a defined amino acid. Due to the degeneracy of the genetic code some amino acids are encoded by more than one codon. These different codons encoding the same amino acid have different relative usage frequencies in individual host cells. Thus, a specific amino acid can be encoded by a group of different codons. Likewise the amino acid sequence of a polypeptide can be encoded by different nucleic acids. Therefore, a specific amino acid can be encoded by a group of different codons, whereby each of these codons has a usage frequency within a given host cell.

As used herein, the term “frameshift mutation” is used herein to mean a genetic mutation caused by a deletion or insertion of a DNA sequence that shifts the way the sequence is read. The insertion or deletion can change the reading frame, resulting in a completely different translation product as compared to a wild-type version of the DNA sequence. For example, the frameshift mutation can be the insertion of N*3+1 base pairs (e.g. +1, +4, +7, etc.), the insertion of N*3+2 base pairs (e.g. +2, +5, +8, etc.), the deletion of N*3-1 base pairs (e.g. −1, −4, −7, etc.), the deletion of N*3-2 basepairs (e.g. −2, −5, −7, etc.) or any combination thereof that leads to an exon length that is not a multiple of 3.

Intron cassettes. Disclosed herein are intron cassettes. The word intron is derived from the terms intragenic region (Gilbert W (February 1978). Nature. 271 (5645): 501), and intracistron (Tonegawa S, Maxam A M, Tizard R, Bernard O, Gilbert W (March 1978). Proceedings of the National Academy of Sciences of the United States of America. 75 (3): 1485-9), that is, a segment of DNA that is located between two exons of a gene. The term intron refers to both the DNA sequence within a gene and the corresponding sequence in the unprocessed RNA transcript. As part of the RNA processing pathway, introns are removed by RNA splicing either shortly after or concurrent with transcription (Tilgner H, Knowles D G, Johnson R, Davis C A, Chakrabortty S, Djebali S, Curado J, Snyder M, Gingeras T R, Guigo R (September 2012). Genome Research. 22 (9): 1616-25). Introns are found in the genes of most organisms and many viruses. They can be located in a wide range of genes, including those that generate proteins, ribosomal RNA (rRNA), and transfer RNA (tRNA) (Roy S W, Gilbert W (March 2006). Genetics. 7 (3): 211-21).

Within introns, a donor site (5′ end of the intron), a branch site (near the 3′ end of the intron) and an acceptor site (3′ end of the intron) are required for splicing. The splice donor site includes an almost invariant sequence GU at the 5′ end of the intron, within a larger, less highly conserved region. The splice acceptor site at the 3′ end of the intron terminates the intron with an almost invariant AG sequence. Upstream (5′-ward) from the AG there is a region high in pyrimidines (C and U), or polypyrimidine tract. Further upstream from the polypyrimidine tract is the branchpoint, which includes an adenine nucleotide involved in lariat formation (Clancy S (2008). Nature Education. 1 (1): 31; and Black D L (June 2003). Annual Review of Biochemistry. 72 (1): 291-336). The consensus sequence for an intron (in IUPAC nucleic acid notation) is: G-G-[cut]-G-U-R-A-G-U (donor site) . . . intron sequence . . . Y-U-R-A-C (branch sequence 20-50 nucleotides upstream of acceptor site) . . . Y-rich-N-C-A-G-[cut]-G (acceptor site) (“Molecular Biology of the Cell”. 2012 Journal Citation Reports. Web of Science (Science ed.). Thomson Reuters. 2013). However, it is noted that the specific sequence of intronic splicing elements and the number of nucleotides between the branchpoint and the nearest 3′ acceptor site affect splice site selection (Taggart A J, DeSimone A M, Shih J S, Filloux M E, Fairbrother W G (June 2012). “Nature Structural & Molecular Biology. 19 (7): 719-21; and Corvelo A, Hallegger M, Smith C W, Eyras E (November 2010). Meyer (ed.). PLoS Computational Biology 6 (11)). Also, point mutations in the underlying DNA or errors during transcription can activate a cryptic splice site in part of the transcript that usually is not spliced. This results in a mature messenger RNA with a missing section of an exon. In this way, a point mutation, which might otherwise affect a single amino acid, can manifest as a deletion or truncation in the final protein.

Cell specific exon sequences. As described in the methods described herein, the cell specific exon sequence can be spliced into the messenger RNA (mRNA) in a cell type of interest. As described in a method described herein, the intron cassette contains “introns” that are spliced out depending on which donor/acceptor sequences are used. In the final mRNA sequence that is derived from the construct, the cell specific exon sequence would remain from the intron cassette in the cell type of interest. Other cells using the constitutive splice donor and constitutive splice acceptor sites would splice out everything, including the cell specific exon sequence. The splicing incorporation of a cell specific exon sequence into messenger RNA results in reading frameshift in the cell type of interest.

In some aspects, the splice donor site can be upstream from the cell specific exon sequence within the intron cassette. In some aspects, the splice donor site can be positioned at the 5′ end of the intron cassette. In some aspects, the splice donor site can be an alternative splice donor site. In some aspects, the splice donor site can be a constitutive splice donor site. In some aspects, the splice acceptor site can be downstream from the cell specific exon sequence within the intron cassette. In some aspects, the splice acceptor site can be positioned at the 3′ end of the intron cassette. In some aspects, the splice acceptor site can be an alternative splice acceptor site. In some aspects, the splice acceptor site can be a constitutive splice acceptor site. In some aspects, the one or more branch sites can be positioned approximate or near to the 3′ end of the intron cassette. In some aspects, the one or more branch sites can flank the cell specific exon sequence. In some aspects, the one or more branch site can be upstream of a splice acceptor site. In some aspects, the one or more branch sites can be positioned upstream, downstream or a combination thereof in relation to the cell specific exon sequence within the intron cassette. For example, as shown in FIG. 11, there can be a branch site upstream of each splice acceptor site. Also as shown in FIG. 11, there can be a branch site upstream of the ASA and a branch site upstream of the CSA. In some aspects, the branch site upstream of the CSA is not associated with the ASA. In some aspects, the one or more branch sites can be positioned upstream, downstream or a combination thereof in relation to the cell specific exon sequence within the intron cassette.

Processing of eukaryotic pre-mRNAs is a complex process that requires a multitude of signals and protein factors to achieve appropriate mRNA splicing. Exon definition by the spliceosome requires more than the canonical splicing signals which define intron-exon boundaries. For example, one such additional signal is provided by cis-acting regulatory enhancer and silencer sequences. Exonic splicing enhancers (ESE), exonic splicing silencers (ESS), intronic splicing enhancers (ISE) and intron splicing silencers (ISS) have been identified which either repress or enhance usage of splice donor sites or splice acceptor sites, depending on their site and mode of action (Yeo et al. 2004, Proc. Natl. Acad. Sci. U.S.A. 101 (44):15700-15705). Binding of specific proteins (trans-acting factors) to these regulatory sequences directs the splicing process, either promoting or inhibiting usage of particular splice sites and thus modulating the ratio of splicing products (Scamborova et al. 2004, MoI. Cell. Biol. 2¥(5j:1855-1869; Hovhannisyan and Carstens, 2005, MoI. Cell. Biol. 25(1):250-263; Minovitsky et al. 2005, Nucleic Acids Res. 330:714-724).

Also described herein are intron cassettes that comprise one or more exon splicing enhancer sequences. Splicing enhancer sequences confer cell specificity during exon splicing. In some aspects, the splicing enhancer sequences are not known. For exon splicing to occur, a cell type of interest must express the specific splicing factors (e.g., proteins that bind to RNA) that can induce the splicing of a cell specific exon sequence (for example, see, Baralle and Giudice, Nat. Rev. Mol. Cell Biol. 2017, 18(7): 437-451). As long as the nucleic construct can be delivered to the target cell or cell type of interest, the splicing machinery in the target cell or cell type of interest will do all the work. In some aspects, the intron cassette can be spliced in a cell type of interest.

In some aspects, the cell specific exon sequence can be flanked by sequences of intron cassette. The sequences of the intron cassette that flank the cell specific exon sequence can be of any length and can be determined by one of ordinary skill in the art. In some aspects, the sequences of the intron cassette can be referred to as a first intron sequence and a second intron sequence such that the first intron sequence is 3′ to the splice donor site (5′ end of the intron), and the second intron sequence is 5′ to the acceptor site (3′ end of the intron). In some aspects, the sequences of the intron cassette that flank the cell specific exon sequence can be 10, 15, 20, 25, 30, 35, 40, 45, 55, 60, 65, 70, 75, 80, 85, 90, 100 or more base pairs in length. The intron sequence on either side of the cell specific exon sequence can be the same or different. As described herein, common splicing elements or sequences (e.g., splicing enhancer sequences) are present in the sequences of the intron cassette that flank the cell specific exon sequence and are responsible for exon splicing. Referring to FIG. 11, while the splice donor sites, branch site (s), and splice acceptor sites (e.g., CSD, ASA, ASD, CSA, and B) are important for the process of exon splicing, other sequences (e.g., exonic splicing enhancers, exonic splicing silencers, intronic splicing enhancers, and intron splicing silencers (ISS)) are important for conferring cell type specificity. In some aspects, CSD and CSA can be replaced by other “common” or “constitutive” splicing donor/acceptor sequences.

In some aspects, the cell specific exon sequence does not comprise a premature stop codon in a canonically spliced reading frame. In some aspects, cell specific exon sequence can be specific for any cell type. Cell specific exon sequences can also be referred to as “alternative exon sequences”, “cell-specific exon sequences” or “cell type-specific alternative exon sequences” that comprise sequence motifs used to distinguish one cell type from another such that exon splicing occurs specifically in a specific cell type.

The term “specific for a cell type” used herein refers to a single cell type that exon splicing occurs based on the cell specific exon sequence. In some aspects, the cell type can be a neuron, a skeletal muscle cell, a cochlear hair cell, an oligodendrocyte or a photoreceptor cell. In some aspects, the cell type can be a stem cell, bone cell, blood cell, muscle cell, fat cell, skin cell, nerve cell, glial cell, endothelial cell, epithelial cell, mesenchymal cell, cells of the immune system, cells of the gastrointestinal tract, cells of the retina, liver cells, exocrine secretory cell, enteroendocrine cell, barrier cell, connective tissue cells, gender specific cells (e.g., sex cells), pancreatic islet cells, or cancer cells. In some aspects, the cell specific exon sequence is spliced in-frame to the start codon upon introducing the nucleic acid construct to the specific cell type.

Promoters. In some aspects, the nucleic acid constructs disclosed herein can further comprise a promoter. The promoter can be any promoter. The promoter can be ubiquitous or cell type specific as the splicing regulation is independent of the promoter. For example, a ubiquitous promoter with a neuron-specific exon sequence can be used to drive gene expression only in neurons. Alternatively, the cell specific promoter can be enhanced, for example, by using a general muscle-specific promoter with a heart-specific exon sequence to drive gene expression only in heart (and not other muscle types). In some aspects, the promoter can be operatively linked to 5′UTR. In some aspects, the promoter can be operatively linked to a start codon. In some aspects, the promoter can be regulatable. In some aspects, the promoter can be constitutively active. In some aspects, the promoter can be constitutively active and drive transcription to levels higher than what is possible with cell specific promoters.

As used herein, the term “promoter” refers to regulatory elements, promoters, promoter enhancers, internal ribosomal entry sites (IRES) and other elements that are capable of controlling expression (e.g., transcription termination signals, including but not limited to polyadenylation signals and poly-U sequences). Promoters can direct constitutive expression. Promoters can also direct expression in a temporal-dependent manner including but not limited to cell-cycle dependent or developmental stage-dependent. Examples of promoters include but are not limited to WPRE, CMV enhancers, and SV40 enhancers. Specific gene specific promoters can be used. Such promoters allow cell specific expression or expression tied to specific pathways. Any promoter that is active in mammalian cells can be used. In some aspects, the promoter is an inducible promoter including, but not limited to, Tet-on and Tet-off systems. Such inducible promoters can be used to control the timing of the desired expression. In some aspects, the promoter can be an inducible promoter. Examples of inducible promoters include but are not limited to tetracycline inducible system (tet); heat shock promoters and IPTG activated promoters. In some aspects, promoters are bidirectional.

The promoter and/or enhancer can be specifically activated either by light or specific chemical events which trigger their function. Systems can be regulated by reagents such as tetracycline and dexamethasone.

In some aspects, the nucleic acid constructs as disclosed herein can comprise a promoter, for example but not limited to, enhancers, 5′ untranslated regions (5′UTR), 3′ untranslated regions (3′UTR), and repressor sequences; constitutive promoters, inducible promoter; tissue specific promoter, cell-specific promoter or variants thereof. Examples of tissue-specific promoters include, but are not limited to, EF1α, CMV, SV40, Ubc, TRE, CAG, PGK1, MND, GAD67, Rho, mDlx, PV, hSyn, CaMKIIα, Nes, Polyhedrin, albumin, lymphoid specific promoters, T-cell promoters, neurofilament promoter, pancreas specific promoters, milk whey promoter; hox promoters, a-fetoprotein promoter, human LIMK2 gene promoters, FAB promoter, insulin gene promoter, transthyretin, al-antitrypsin, plasminogen activator inhibitor type 1 (PAI-1), apolipoprotein myelin basic protein (MBP) gene, GFAP promoter, OPSIN promoter, NSE, Her2, erb2, and fragments and derivatives thereof. Examples of other promoters include, but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and variants thereof.

Gene of interest. In some aspects, the nucleic acid constructs disclosed herein can further comprise a gene of interest. In some aspects, the gene of interest is downstream of the intron cassette. In some aspects, the gene of interest is in-frame with the reading frame after the cell specific exon is spliced in. In some aspects, the gene of interest can be a therapeutic agent or a detectable moiety. Examples of detectable moieties include but are not limited to fluorescein for fluorescence, HA tag, Gst-tag, EGFP-tag, FLAG™ tag or biotin. In some aspects, the therapeutic agent can be an enzyme, a hormone, a polypeptide, an antibody, a drug, a chemotherapeutic agent, a toxin, or an oligonucleotide.

The compositions generated herein comprising a gene of interest can be used to monitor and/or modulate cell activity. In some aspects, the compositions described herein can be designed to comprise an effector molecule. The term “effector molecule” can be used herein to refer to a small molecule that selectively can bind to a protein and regulate its biological activity. In some aspects, effector molecules can act as ligands. In some aspects, effector molecules can increase or decrease enzyme activity, gene expression or cell signaling. In some aspects, the effector molecules can be calcium sensors (e.g., GCaMP7), channelrhodopsin-2 (ChR2) or Designer Receptors Exclusively Activated by Designer Drugs (DREADDS).

DREADDS. In some aspects, the gene of interest can encode for a modified receptor such that it can be modified to be solely activated by artificial or exogenous agonists, referred to herein as a “modified receptor” or a “DREADD”. Receptors modified in this way are known to one of ordinary skill in the art using a technology called Designer Receptors Exclusively Activated by Designer Drugs (DREADD). A receptor can be modified such that it is mutated to render it insensitive to endogenous ligands but sensitive to a substance that normally has no effect. One of ordinary skill in the art can provide or design such a modified receptor using known methods, and in view of the instant disclosure, apply them to the compositions and methods disclosed herein. The terms “modified receptor” and “DREADD” can be used interchangeably. A modified receptor (e.g., GPCR, PAR) can have a decreased binding affinity for a selected natural (e.g., endogenous) ligand of the modified receptor (relative to binding of the selected ligand by a wild-type receptor (e.g., GPCR, PAR)), but having normal, near normal, or preferably enhanced binding affinity for an exogenous, typically synthetic, ligand (e.g., a peptide or small molecule). Thus, modified receptor-mediated activation of modified receptor-expressing cells does not occur to a significant extent in vivo in the presence of the natural ligand, but responds significantly upon exposure to an exogenously introduced ligand (e.g., agonist). For example, the modified receptor can be superiorly activated by an exogenous ligand as compared to the natural ligand (e.g., activated to a greater or more significant extent by binding of the ligand than by binding to a selected natural or endogenous ligand at a similar concentration).

“Natural ligand” and “naturally occurring ligand” and “endogenous ligand” of a native GPCR can be used interchangeably herein to mean a biomolecule endogenous to a mammalian host, wherein the biomolecule binds to a native GPCR to elicit a G protein-coupled cellular response.

“Synthetic small molecule, “synthetic small molecule ligand,” “synthetic ligand”, “synthetic agonist”, “exogenous agonist”, exogenous ligand” and the like are used interchangeably herein to mean any compound made exogenously by natural or chemical means that can bind within the transmembrane domains of a G protein-coupled receptor or modified G protein-coupled receptor or modified PAR (i.e., DREADD) and facilitate activation of the receptor and concomitant activation of a desired family of G proteins.

As used herein the term “binding” can be used interchangeably with the terms “receptor-ligand binding” or “ligand binding,” to mean physical interaction between a receptor (e.g., a G protein-coupled receptor or a modified receptor) and a ligand (e.g., a natural ligand, (e.g., peptide ligand) or synthetic ligand (e.g., synthetic small molecule ligand)). Ligand binding can be measured by a variety of methods known in the art (e.g., detection of association with a radioactively labeled ligand).

In some aspects, the modified receptor can be a modified G-protein coupled receptor (GPCR). In some aspects, the modified GPCR can be a Gq, a Gi, a Gs or a G₁₂/G₁₃ receptor.

“G protein-coupled receptor” as used herein refers to a receptor that, upon binding of its natural ligand and activation of the receptor, transduces a G protein-mediated signal(s) that results in a cellular response. G protein-coupled receptors form a large family of evolutionarily related proteins. Proteins that are members of the G protein-coupled receptor family are generally composed of seven putative transmembrane domains. G protein coupled receptors were also known in the art as “seven transmembrane segment (7TM) receptors” and as “heptahelical receptors”. GPCRs detect molecules outside the cell and activate internal signal transduction pathways and, ultimately, cellular responses. GPCRs interact with a complex of heterotrimeric guanine nucleotide-binding proteins (G-proteins) and thus regulate a wide variety of intracellular signaling pathways including ion channels. For example, when a ligand binds to the GPCR it causes a conformational change in the GPCR, which allows it to act as a guanine nucleotide exchange factor (GEF). The GPCR can then activate an associated G protein by exchanging the GDP bound to the G protein for a GTP. The G protein's α subunit, together with the bound GTP, can then dissociate from the β and γ subunits to further affect intracellular signaling proteins or target functional proteins directly depending on the a subunit type (Gαs, Gαi/o, Gαq/11, Gα12/13). As used herein, a “G protein-coupled cellular response” or “GPCR cellular response” means a cellular response or signaling pathway that occurs upon ligand binding by a GPCR. Such GPCR cellular responses relevant to the present disclosure are those which trigger the activation of one or more modified receptors which in turn can inhibit or stimulate a particular circuit. The type of response whether it is an inhibitory response or excitatory response will depend on the type of GPCR activated.

The term “signaling” as used herein can mean the generation of a biochemical or physiological response as a result of ligand binding (e.g., as a result of synthetic or exogenous ligand binding to a modified receptor).

The terms “receptor activation,” “DREADD activation,” “modified receptor activation”, “GPCR activation”, and “PAR activation” can be used interchangeably herein to mean binding of a ligand (e.g., a natural or synthetic ligand) to a receptor in a manner that elicits G protein-mediated signaling, and a physiological or biochemical response associated with G protein-mediated signaling. Activation can be measured by measuring a biological signal associated with G protein-related signals.

“Targeted cellular activation” and “target cell activation” can be used interchangeably herein to mean DREADD-mediated activation or receptor-mediated activation of a specific G protein-mediated physiological response in a target cell, wherein DREADD-mediated activation or receptor-mediated activation occurs by binding of an endogenous ligand molecule to the DREADD or modified receptor.

The compositions and methods described herein can affect or elicit G protein-mediated cellular response of any eukaryotic cell.

In some aspects, the modified receptor can be a modified G-protein coupled receptor (GPCR). In some aspects, the modified GPCR can be a Gq, a Gi, a Gs or a G₁₂/G₁₃ GPCR.

Cells. Disclosed herein are cells comprising any of the nucleic acid constructs described herein. In some aspects, the cell can be a specific cell. In some aspects, the cell can be a eukaryotic cell. In some aspects, the eukaryotic cell can be a mammalian cell. In some aspects the cell can be a specific eukaryotic cell. In some aspects, the cell can be a specific mammalian cell. In some aspects, the cell can be a cell within a subject. In some aspects, the cell can be a neural, a skeletal muscle cell, a cochlear hair cell, an oligodendrocyte or a photoreceptor cell. In some aspects, the cell can be a stem cell, bone cell, blood cell, muscle cell, fat cell, skin cell, nerve cell, endothelial cell, connective tissue cells, gender specific cells (e.g., sex cells), pancreatic cells, or cancer cells. In some aspects, the cell can be a diseased cell. In some aspects, the eukaryotic cell can be a diseased cell. In some aspects, the mammalian cell can be a diseased cell.

In some aspects, the nucleic acid constructs as described herein can be delivered to a cell of a subject.

Vectors. Disclosed herein are vectors comprising any of the nucleic acid constructs described herein. Vectors comprising nucleic acids or polynucleotides as described herein are also provided. As used herein, a “vector” refers a carrier molecule into which another DNA segment can be inserted to initiate replication of the inserted segment. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, and viruses (e.g., bacteriophage, animal viruses, and plant viruses), and artificial chromosomes (e.g., YACs). Vectors can comprise targeting molecules. A targeting molecule is one that directs the desired nucleic acid to a particular organ, tissue, cell, or other location in a subject's body. A vector, generally, brings about replication when it is associated with the proper control elements (e.g., a promoter, a stop codon, and a polyadenylation signal). Examples of vectors that are routinely used in the art include plasmids and viruses. The term “vector” includes expression vectors and refers to a vector containing a nucleic acid sequence coding for at least part of a gene product capable of being transcribed. A variety of ways can be used to introduce an expression vector into cells. In some aspects, the expression vector comprises a virus or an engineered vector derived from a viral genome. As used herein, “expression vector” is a vector that includes a regulatory region. A variety of host/expression vector combinations can be used to express the nucleic acid sequences disclosed herein. Examples of expression vectors include but are not limited to plasmids and viral vectors derived from, for example, bacteriophages, retroviruses (e.g., lentiviruses), and other viruses (e.g., adenoviruses, poxviruses, herpesviruses and adeno-associated viruses). Vectors and expression systems are commercially available and known to one skilled in the art.

The vectors disclosed herein can also include detectable label or selectable marker. Such detectable labels can include a tag sequence designed for detection (e.g., purification or localization) of an expressed polypeptide. Tag sequences include, for example, green fluorescent protein, glutathione S-transferase, polyhistidine, c-myc, hemagglutinin, or Flag™ tag, and can be fused with the encoded polypeptide and inserted anywhere within the polypeptide, including at either the carboxyl or amino terminus.

The term “expression cassette” as used herein refers to a nucleic acid construct. The expression cassette can be produced either through recombinant techniques or synthetically that will result in the transcription of a certain polynucleotide sequence in a host cell. The expression cassette can be part of a plasmid, viral genome or nucleic acid fragment. Generally, the expression cassette includes a polynucleotide operably linked to a promoter. In some aspects, the expression cassette can be a plasmid. Plasmids that are useful include pAAV-mDlx-GFP, pAAV-CAG-GFP, pX601-AAV-CMV, pLv-HSA-uDys/eGFP, AAV-pgk-Cre, pAAV-hSyn-DIO-mCherry, pAAV-Ef1a-mCherry-IRES-Cre, pAAV-FLEX-GFP, pAAV-minCMV-mCherry, and pAAV-CaMKIIa-hChR2(H134R)-EYFP. Examples of AAV vectors are known (https://www.addgene.org/viral-vectors/aav/). The expression cassette can be adapted for expression in a specific type of host cell (e.g., using a cell specific exon sequence). The expression cassette can also comprise other components such as polyadenylation signals, enhancer elements or any other component that results in the expression of the nucleic acid constructs disclosed herein in a specific type of host cell.

Vectors include, for example, viral vectors (such as adenoviruses (“Ad”), adeno-associated viruses (AAV), and retroviruses, including lentiviruses), liposomes and other lipid-containing complexes, and other macromolecular complexes capable of mediating delivery of a polynucleotide to a host cell. Vectors can also comprise other components to further modulate the delivery and/or expression of the gene of interest, for example, or that otherwise provides beneficial properties to the targeted cells. A wide variety of vectors is known to those skilled in the art and is generally available. Other suitable complexes capable of mediating delivery of any of the nucleic acid constructs described herein include retroviruses (e.g., lentivirus), vaults, cell penetrating peptides and biolistic particle guns. Cell penetrating peptides are capable of transporting or translocating proteins across a plasma membrane; thus, cell penetrating peptides act as delivery vehicles. Examples include but are not limited to labels (e.g., GFP, MRI contrast agents, quantum dots).

TABLE 1
Sequences.

| SEQ | | |
|---|---|---|
| ID NO: | Name | Sequence |
| 1 | Nucleic acid construct | ATGAGATGGGCCAACTTCCATCTGGAAAACTCAGGCTGGCAAAAGATCAACAA |
| | neuron specific | CTTCAGCGCTGACATCAAGgtaactgtgcaaaaaaccatagcgttcaggtaga |
| | | tacgatggcttcagggattttatcccattgtaacagagacaaaaatgatagga |
| | | tatcaccaggccctgttgtctttccctaggagctaatttccttaagtatattc |
| | | tgattttaaaaggttaaatgctctcttgccaaccatatgtgtttctcgtttaa |
| | | gcttttctgtatttcaacttttgatttaacatacaattaacatagtagatgtt |
| | | gtttccataggttgctactcatcatgctttgtaatgccataatgtgttcattt |
| | | tctctttccctatgcttcctggatttctgctcttctatttcatgcttgtttat |
| | | ctgtcaagCTTATTGACTTCAGTAA[C]TTCAGTGAAGgtacagaaatactta |
| | | atagctttattgggttgatggcatcgataaattgtcaagtcctaacaatcacg |
| | | tgcaaagacaaaaagaaagatctctcaaataagaagacagagaagaatgcctt |
| | | atattatcacaaaagctgctcccaatgtcctcatcaccaagggacaataagat |
| | | gcagctactctttggcagtgtgatcacactattatgttcaattcttttgtagG |
| | | ATTCCAAAGCCTATTTCCATCTGCTCAATCAAATCGCACCGAAGGGA |
| 2 | Sequence yielded in | ATGAGATGGGCCAACTTCCATCTGGAAAACTCAGGCTGGCAAAAGATCAACAA |
| | most cells after splicing | CTTCAGCGCTGACATCAAGGATTCCAAAGCCTATTTCCATCTGCTCAATCAAA |
| | using SEQ ID NO: 1 | TCGCACCGAAGGGA |
| 3 | Sequence yielded in | ATGAGATGGGCCAACTTCCATCTGGAAAACTCAGGCTGGCAAAAGATCAACAA |
| | neurons after splicing | CTTCAGCGCTGACATCAAGCTTATTGACTTCAGTAA[C]TTCAGTGAAGGATT |
| | using SEQ ID NO: 1 | CAAAGCCTATTTCCATCTGCTCAATCAAATCGCACCGAAGGGA |
| 4 | Nucleic acid construct | ATGATCAACTTCTATGCAGGGGCAAACCAGAGCATGAATGTCACTTGTGTTGG |
| | photoreceptor specific | CAAGgtgagtgtgggggccctccttacctgcccacctggttagacttcctggt |
| | | ttctgagtgcttcacccatatctccctatctttttgtgctttcagAGGCCACA |
| | | G[T]CACTA{G}AGGGACAAGGGGgtaagagtgggcgcctatgcagttttagc |
| | | tctaagaggctcttagccctattgcttctctctaggataaagagagcctgctg |
| | | tcctggagatagacctatcccttcctgcaccaaagctctgacctctggttcct |
| | | tccctgtcaactttttcttacatctcagttgtctgggtttcttccactctccc |
| | | atcatgccttgtttctcagttccctcagtctgctagctactgctcagttagca |
| | | ccctttgctacaactagttgtccttggaaccctgcagccaactctgtcctctc |
| | | tagaaactctcctccttcccactgagccttgactgtttatctgttctttcttg |
| | | gctctgctccagagactgattcccaaggacggggtaagaacttggggattgat |
| | | ggtggagttagaaggccctcaccgtgttgtcagcacccttagaagacctagtc |
| | | tgatgggagataggccacccctatctgcagacatgcagataggaacatgtgtg |
| | | catgcgcacacacaaatgcacacacagctacctgagcagatgcacagctcaaa |
| | | gaaaacaagtttgacagggataatttgggatgaaggaggtacagaaggaagtc |
| | | ttgtgagcgcttccaggtgcctgctgttcctaacatcctctccccttaacctt |
| | | cctgcacccccagagAGAGAGAAGATGCTGAGAACCTTGGCCACTTTGTCATG |
| | | TTCCCTGCTAATGGCAGCA |
| 5 | Sequence yielded in | ATGATCAACTTCTATGCAGGGGCAAACCAGAGCATGAATGTCACTTGTGTTGG |
| | most cells after splicing | CAAGAGAGAGAAGATGCTGAGAACCTTGGCCACTTTGTCATGTTCCCTGCTAA |
| | using SEQ ID NO: 4 | TGGCAGCA |
| 6 | Sequence yielded in | ATGATCAACTTCTATGCAGGGGCAAACCAGAGCATGAATGTCACTTGTGTTGG |
| | photoreceptors after | CAAGAGGCCACAG[T]CACTA{G}AGGGACAAGGGGAGAGAGAAGATGCTGAG |
| | splicing using | AACCTTGGCCACTTTGTCATGTTCCCTGCTAATGGCAGCA |
| | SEQ ID NO: 4 | |
| 7 | Nucleic acid construct | ATGTGCAAGAAAAGAAAAGGTCAAGCATCTGGCAATTgtgagtgagggccggg |
| | muscle cell specific | gtcagacaagagcgttaccctgacctttcccagtgggaggagatagctaaact |
| | | gaagttcttctctttaatatacagCATGCCAACTC[G]Ggtaaggctgtgctg |
| | | caggacccaaagtaggctgtcgtaagctgcactgtgttggttatgcagtggct |
| | | ggactcttctgctggtgtacataaaaccatggggaaatggttctaaaattgac |
| | | agtctcaggaatcctcaccaaagggatgaggaaaagtgtagtatttgtactgt |
| | | ttggttagttgtcttgtatgtgttaaacctgactttgtaccagaagtactgtg |
| | | tgtttattgaagttaaaacttatccttttaagattaatctgtttactggaagc |
| | | ataaatgttttaaaactgtggacatgaaaatagttccttaagttgagcttaga |
| | | aaattcatgctatgagttaacttggtgatgtgtgttctcagtgtagcagacct |
| | | ttgtatgcgctgcacactaagctttggctgttatcacggtcatccccctgctg |
| | | ctcactgaccactgtgactttgtcccacataggacctagctagccaaggagcc |
| | | ttcgtattgtacttgtttgtaggagctttcagtacgcaagagcatctctttca |
| | | ttatggaacttgcttatatctagcattctaagcggtagctattcccccgagtg |
| | | tccaagtttggttgtgactgactgctaaggcgatagtataaccataaagctgg |
| | | ttttgctaatgtctaactcactgccttgtaaagttttgaaattttcttgatta |
| | | taaaaatttatggaaaattggggcttggttttcacaatgcatgtaataataga |
| | | catatctgtgggttttgtttgcctcttttattttgttagTTTCAGCCGACTTT |
| | | TTAGCTCTTCAAGTAATGCAACAAAGAAGCCTGAAC |
| 8 | Sequence yielded in | ATGTGCAAGAAAAGAAAAGGTCAAGCATCTGGCAATTTTTCAGCCGACTTTTT |
| | most cells after splicing | AGCTCTTCAAGTAATGCAACAAAGAAGCCTGAAC |
| | using SEQ ID NO: 7 | |
| 9 | Sequence yielded in | ATGTGCAAGAAAAGAAAAGGTCAAGCATCTGGCAATTCATGCCAACTC[G]GT |
| | muscle cells after | TTCAGCCGACTTTTTAGCTCTTCAAGTAATGCAACAAAGAAGCCTGAAC |
| | splicing using | |
| | SEQ ID NO: 7 | |
| 10 | Nucleic acid construct | ATGGATCAAAAACGCTCAGGAGCTGGCTTGTGGAGTGTGTCTCTTAAATGTGG |
| | glutamatergic specific | ACTCCAGGAGCCGGgtaagtcctggccttcctccttcaagggccattgtttat |
| | | ccctaacatgatttaaggggcagccacatgtagcccccttcacaagtgctgtg |
| | | accagaggtggggatgctgaagtctagaaaggtctatagaagcagatctgttt |
| | | tttccacagggcagtggggaactgctgagggattaaacagagtcctccctccc |
| | | tgctgagcattggattcacccgggacaggggtgggtggagccagaaggccagg |
| | | ccctgtggacttacctggaggcaagagccgttccaggggtttgattagctttt |
| | | tcttttgaccttatggtttgaacatttcagAAAGAAGAGACGCCTGCAGAA |
| | | [G]GAACAGCCTAAAAAAgtatgagccagctacatgttactttagtgtctcca |
| | | tctcagtcttgtgtgtgagaggccaacagataattctgttgttgtcctggact |
| | | gtaccagttgtgaatcctctgctgactcactcgtccccataacgtgtgatcaa |
| | | atcaactaatgcgtctctgtggggccactgccgtcctgtttgcgtgcttgtag |
| | | ctgctaatcagcttctgctgttgtccgtatctttcctcaccacgaatcagcac |
| | | cttctggaagactagaagttgtagtgacagagaatgagatggcagagcgtcag |
| | | gatttagtggatgagagacaaaaaaagaaaaaaatcaaatcatttgggttcta |
| | | ggattttaactgtagtgtttaagaaaacaaactgcaggctgttacctgctgtg |
| | | ggtagctgaaggagggtctcatatgtccactcagttgtcagcccccaatggca |
| | | gtggaccagtgattctcaaccttcctgatgctgctgcgaccctttaatacagt |
| | | tcttcatggtgtgttgacccccaagcataaaattattttgtgggtaccgtaat |
| | | ttcgctgctattagaagtcgtaatgtaaatacctggcatgggtcctgactcac |
| | | aggttgagaactgctgcatgagactttaagagttaatatcaattgatttctaa |
| | | ctcttttaaatgttttcagtcctgatagaactgtaattagccacagacctaag |
| | | gggccacatgttacagcagtggcagcctccagggagggtggctattatagtga |
| | | gggaaccaattacccacaggccctggctattttgattcccgtgccctgcccct |
| | | ttttctctcctgtaacctctgcctgccttttctgtgaagattggcccttttgc |
| | | tgaaactaggttggatggtactactgactttatcctttaattagcagtaaaaa |
| | | tgaataaaactttcctacttggatcctgtagccacctgctcggcttttgtctg |
| | | tgtgtgccatttgggcctcagccttggtgacacctggggagtgtccgtagtgg |
| | | ttgctgacaagctctcagatgtagaaagaccctgacttgcccccatctctcta |
| | | tttctggcagGCATTCAACTCGGAGACAGACAGCTTCAAGCTGGCCTACGGAG |
| | | GACACCAGTACCA |
| 11 | Sequence yielded in | ATGGATCAAAAACGCTCAGGAGCTGGCTTGTGGAGTGTGTCTCTTAAATGTGG |
| | GABAergic neurons | ACTCCAGGAGCCGGGCATTCAACTCGGAGACAGACAGCTTCAAGCTGGCCTAC |
| | after splicing using | GGAGGACACCAGTACCA |
| | SEQ ID NO: 10 | |
| 12 | Sequence yielded in | AGTGATCAAAAACGCTCAGGAGCTGGCTTGTGGAGTGTGTCTCTTAAATGTGG |
| | glutamatergic neurons | ACTCCAGGAGCCGGAAAGAAGAGACGCCTGCAGAA[G]GAACAGCCTAAAAAA |
| | after splicing using | GCATTCAACTCGGAGACAGACAGCTTCAAGCTGGCCTACGGAGGACACCAGTA |
| | SEQ ID NO: 10 | CCA |

ATG = Start Codon and UNDERLINED NON-BOLDED SEQUENCES indicate the flanking sequences of constitutive
exons. Lowercase letters indicate spliced out intronic sequences.

SEQ ID NO: 1: neuronal intron sequence with neuron-specific exon sequence bolded and underlined ([C] nucleotide is the +1 insertion mutation added to shift the reading frame).

SEQ ID NO: 4: photoreceptor intron sequence with photoreceptor-specific exon bolded and underlined ([T] nucleotide is the +1 insertion mutation added to shift the reading frame, {G} nucleotide is a point mutation intended to remove premature stop codon).

SEQ ID NO: 7: muscle intron sequence with muscle-specific exon bolded and underlined ([G] nucleotide is the +1 insertion mutation).

SEQ ID NO: 10: glutamatergic intron sequence with glutamatergic-specific exon sequence bolded and underlined ([G] nucleotide is the +1 insertion mutation).

### Methods

Disclosed herein are methods of expressing a nucleotide sequence in a specific cell. In some aspects, the methods can comprise introducing any of the nucleic acid constructs disclosed herein to the specific cell. In some aspects, the specific cell can be a eukaryotic cell. In some aspects, the eukaryotic cell can be a mammalian cell. In some aspects the specific cell can be a specific eukaryotic cell. In some aspects, the specific eukaryotic cell can be a specific mammalian cell. In some aspects, the methods disclosed herein can be carried out in any species that is capable of using alternative splicing (e.g., worm, fly, yeast). In some aspects, the cell type can be a neuron, a skeletal muscle cell, a cochlear hair cell, an oligodendrocyte or a photoreceptor cell. In some aspects, the cell type can be a stem cell, bone cell, blood cell, muscle cell, fat cell, skin cell, nerve cell, endothelial cell, connective tissue cells, gender specific cells (e.g., sex cells), pancreatic cells, or cancer cells. In some aspects, the specific cell can be a diseased cell. In some aspects, the eukaryotic cell can be a diseased cell. In some aspects, the mammalian cell can be a diseased cell.

Disclosed herein are methods of treating a human patient. In some aspects, the methods can comprise administering any of the nucleic acid constructs described herein. In some aspects, the human patient has been identified as being in need of treatment before the administration step. In some aspects, the human patient can have a disease or a disorder. In some aspects, the disease or disorder can be a monogenic disease or disorder. A monogenetic disease or disorder are caused by a mutation in a single gene. In some aspects, the monogenetic disease is one in which a protein is dysfunctional in a single cell type. Examples of monogenic diseases or disorder include but are not limited to sickle cell disease, cystic fibrosis, polycystic kidney disease and Tay-Sachs disease. In some aspects, the disease or disorder can be a motor neuron disease or a neurodegenerative disease. In some aspects, the disease or disorder can be Alzheimer's disease, Bell's palsy, cerebral palsy, epilepsy, multiple sclerosis, neurofibromatosis, or Parkinson's disease. In some aspects, the disease or disorder can be a skeletal muscle cell disease. In some aspects, the disease or disorder can be muscular dystrophy. In some aspects, the disease or disorder can be a disease or disorder associated with a cochlear hair cell. In some aspects, the disease or disorder can be associated with an oligodendrocyte. In some aspects, the disease or disorder can be RETINA: retinitis pigmentosa, age-related macular degeneration, choroideremia, achromatopsia, glaucoma, diabetic retinopathy, retinoblastoma, BRAIN: Alzheimer's disease, frontotemporal dementia, vascular dementia, Lewy body dementia, Parkinson's disease, Huntington's disease, amyotrophic lateral sclerosis, autism spectrum disorder, schizophrenia, epilepsy, stroke and transient ischemic attack, traumatic brain injury, glioblastoma, Creutzfeldt-Jakob disease, Charcot-Marie-Tooth disorders, multiple sclerosis, dystonia, neuralgia, seizures, brain hemorrhage, Meniere's disease, Friedreich's ataxia, Gaucher disease, Guillain-Barre syndrome, leukodystrophies, myasthenia gravis, peripheral neuropathy, Angelman Syndrome, Prader-Willi syndrome, progressive supranuclear palsy, Rett syndrome, Tay-Sachs disease, transverse myelitis, multi system atrophy, Sydenham's chorea, Spasmodic dysphonia, MUSCLE: rhabdomyosarcoma, muscular dystrophy, congenital myopathy, hypotonia, myotonia, sarcopenia, sarcoglycanopathy, osteopetrosis, mitochondrial myopathy, HEARING: auditory neuropathy, vestibular neuritis, BPPV, Meniere's disease, presbystasis, Usher syndrome, Treacher Collins syndrome, Crouzon syndrome, Alport syndrome, Waardenburg syndrome, congenital hearing loss, OTHER (general): cancer (e.g. Myelodysplastic syndromes, Acute Myeloid Leukemia, Adrenocortical carcinoma, Bladder Urothelial Carcinoma, Brain Lower Grade Glioma, Breast invasive carcinoma, Cervical squamous cell carcinoma and endocervical adenocarcinoma, Cholangiocarcinoma, Chronic Myelogenous Leukemia, Colon adenocarcinoma, Esophageal carcinoma, Glioblastoma multiforme, Head and Neck squamous cell carcinoma, Kidney Chromophobe, Kidney renal clear cell carcinoma, Kidney renal papillary cell carcinoma, Liver hepatocellular carcinoma, Lung adenocarcinoma, Lung squamous cell carcinoma, Lymphoid Neoplasm Diffuse Large B-cell Lymphoma, Mesothelioma, Miscellaneous, Ovarian serous cystadenocarcinoma, Pancreatic adenocarcinoma, Pheochromocytoma and Paraganglioma, Prostate adenocarcinoma, Rectum adenocarcinoma, Sarcoma, Skin Cutaneous Melanoma, Stomach adenocarcinoma, Testicular Germ Cell Tumors, Thymoma, Thyroid carcinoma, Uterine Carcinosarcoma, Uterine Corpus Endometrial Carcinoma, Uveal Melanoma), heart disease (e.g., congestive heart failure, chronic heart failure, coronary artery disease, critical limb ischemia, myocardial infarction, ischemia, peripheral artery disease, peripheral vascular disease, cardiomyopathy, atrial fibrillation), diabetes mellitus, pulmonary diseases, cirrhosis, anemia, cytopenia, monogenic disorders (e.g. sickle cell disease, cystic fibrosis, polycystic kidney disease, spinal muscular atrophy, al-antitrypsin deficiency, primary ciliary dyskinesia, Alpha-1 Antitrypsin Deficiency, Alexander's Disease, Citrullinemia, Glycogen Storage Diseases, Phenylalanine Hydroxylase Deficiencies, Ornithine-Transcarbamylase Deficiency, Beta Galactosidase 1 Deficiency, Arginosuccinate Synthetase Deficiency, Phenylketonuria, Tyrosinemia, Mucopolysaccharidosis, Menkes Disease, Lysosomal Storage Disorder, Hemophilia, X Chromosome Disorders, Canavan Disease, Batten Disease, Niemann-Pick Disease, Pompe Disease, Fabry Disease, Crigler-Najjar Syndrome, Methylmalonic Acidemia, Lipodystrophy, Hyperlipidemia, Hypercholesterolemia, Thalassemia, Wilson Disease, Kartagener disease, chronic granulomatous disease, respiratory distress syndrome, etc.).

Also disclosed are methods of delivering a therapeutic agent to one or more cells. In some aspects, the methods comprise: contacting the one or more cells with any of the nucleic acid constructs disclosed herein that comprises a gene of interest or a therapeutic agent. In some aspects, the one or more cells can be a specific cell. In some aspects, the specific cell can be a eukaryotic cell. In some aspects, the eukaryotic cell can be a mammalian cell. In some aspects the specific cell can be a specific eukaryotic cell. In some aspects, the specific eukaryotic cell can be a specific mammalian cell. In some aspects, the methods disclosed herein can be carried out in any species that is capable of using alternative splicing (e.g., worm, fly, yeast). In some aspects, the cell type can be a neuron, a skeletal muscle cell, a cochlear hair cell, an oligodendrocyte or a photoreceptor cell. In some aspects, the cell type can be a stem cell, bone cell, blood cell, muscle cell, fat cell, skin cell, nerve cell, glial cell, endothelial cell, epithelial cell, mesenchymal cell, cells of the immune system, cells of the gastrointestinal tract, cells of the retina, liver cells, exocrine secretory cell, enteroendocrine cell, barrier cell, connective tissue cells, gender specific cells (e.g., sex cells), pancreatic islet cells, or cancer cells. In some aspects, the specific cell can be a diseased cell. In some aspects, the eukaryotic cell can be a diseased cell. In some aspects, the mammalian cell can be a diseased cell.

Also disclosed herein are methods of selectively inducing exon splicing in a cell. In some aspects, the methods can comprise contacting a cell with any of the nucleic acid constructs disclosed herein. In some aspects, the one or more cells can be present in a subject. In some aspects, the one or more cells can be a specific cell. In some aspects, the specific cell can be a eukaryotic cell. In some aspects, the eukaryotic cell can be a mammalian cell. In some aspects the specific cell can be a specific eukaryotic cell. In some aspects, the specific eukaryotic cell can be a specific mammalian cell. In some aspects, the methods disclosed herein can be carried out in any species that is capable of using alternative splicing (e.g., worm, fly, yeast). In some aspects, the cell type can be a neuron, a skeletal muscle cell, a cochlear hair cell, an oligodendrocyte or a photoreceptor cell. In some aspects, the cell type can be a stem cell, bone cell, blood cell, muscle cell, fat cell, skin cell, nerve cell, glial cell, endothelial cell, epithelial cell, mesenchymal cell, cells of the immune system, cells of the gastrointestinal tract, cells of the retina, liver cells, exocrine secretory cell, enteroendocrine cell, barrier cell, connective tissue cells, gender specific cells (e.g., sex cells), pancreatic islet cells, or cancer cells. In some aspects, the specific cell can be a diseased cell. In some aspects, the eukaryotic cell can be a diseased cell. In some aspects, the mammalian cell can be a diseased cell.

Methods of activating a modified receptor using DREADD. As disclosed herein, the nucleic acids constructs can comprise a sequence that encodes a modified receptor. As disclosed herein, the nucleic acid constructs can be introduced into a cell. In some aspects, the one or more cells can express the modified receptor. In some aspects, once one or more cells express the modified receptor, the one or more cells can be contacted in the presence of an exogenous agonist. In some aspects, the presence of the exogenous agonist can activate the modified receptor thereby activating or inhibiting a cellular circuit. In some aspects, the exogenous agonist can be administered to a subject or patient in need thereof. In some aspects, the exogenous agonist can be administered before, during or after the delivery of the nucleic acid construct. In some aspects, the exogenous agonist can be administered via intracranial, intraspinal, intramuscular, or intravenous injection or orally. In some aspects, the exogenous ligand or agonist can be selective for or specific to the modified receptor present on a specific cell type. In some aspects, the human patient has been identified as being in need of treatment before the administration step. In some aspects, the human patient can have a disease or a disorder.

Diseases and disorders. In some aspects, the disease can be a monogenic disease. Examples of monogenic diseases or disorder include but are not limited to sickle cell disease, cystic fibrosis, polycystic kidney disease and Tay-Sachs disease. In some aspects, the disease or disorder can be a motor neuron disease or a neurodegenerative disease. In some aspects, the disease or disorder can be Alzheimer's disease, Bell's palsy, cerebral palsy, epilepsy, multiple sclerosis, neurofibromatosis, or Parkinson's disease. In some aspects, the disease or disorder can be a skeletal muscle cell disease. In some aspects, the disease or disorder can be muscular dystrophy. In some aspects, the disease or disorder can be a disease or disorder associated with a cochlear hair cell. In some aspects, the disease or disorder can be associated with an oligodendrocyte. In some aspects, the disease or disorder can be retinitis pigmentosa

In some aspects, the disease can be a cancer. In some aspects, the cancer can be a primary or secondary tumor. In some aspects, the cancer has metastasized. In some aspects, the cancer can be a solid cancer or a blood cancer. The cancer can be any cancer. In some aspects, the cancer can anal cancer, bladder cancer, brain cancer, bone cancer, breast cancer, cervical cancer, colorectal cancer, endocrine cancer, esophageal cancer, eye cancer, gallbladder cancer, head and neck cancer, kidney cancer, leukemia, liver cancer, lymphoma, melanoma, oral or oropharyngeal cancer, osteosarcoma, parathyroid cancer, pancreatic cancer, penile cancer, pituitary gland cancer, prostate cancer, skin cancer, stomach cancer, testicular cancer, thyroid cancer, uterine cancer, vulvar cancer, ovarian cancer, lung cancer, or gastric cancer.

Agonists and Administration. As disclosed herein, the nucleic acids constructs can comprise a sequence that encodes a modified receptor. As disclosed herein, the nucleic acid constructs can be introduced into a cell. In some aspects, the one or more cells can express the modified receptor. In some aspects, once one or more cells express the modified receptor, the one or more cells can be contacted in the presence of an exogenous agonist.

Disclosed herein are modified receptors that can be activated by the presence of an exogenous agonist. The exogenous agonist (or ligand, or small molecule, the terms are used interchangeably herein) is one which can be delivered orally or parenterally (e.g., systemically administered). The ligand is exogenous in that it is generally absent from the body or area to be treated or is present in sufficiently low basal concentrations that it does not activate the modified receptor. In some aspects, the ligand can be synthetic, i.e., not naturally occurring. In some aspects, the ligand is one that possesses minimal or no biologic activity other than DREADD activation or modified receptor activation.

Any small molecule, generally a synthetic small molecule that can bind within the transmembrane domains of the DREADD or modified receptor and facilitate DREADD-mediated activation or modified receptor-mediated activation of a desired family of G proteins is suitable for use in the method described herein. In contrast to the natural peptide ligands of G protein-coupled receptors which typically have molecular weights of 2000-6000 Da, in some aspects, small molecule ligands of G protein-coupled receptors will generally have molecular weights of 100-1000 Da.

Synthetic small molecules useful in the methods disclosed herein include synthetic small molecules generated by either a natural (e.g., isolated from a recombinant cell line) or chemical means (e.g., using organic or inorganic chemical processes).

Several synthetic small molecules that bind and activate native GPCRs are known in the art and can be useful in the methods disclosed herein. Additional synthetic small molecules suitable for use in the methods disclosed herein can be identified by screening candidate compounds for binding to native GPCRS or to DREADDs. For example, by using-a cell line expressing (or transfected with) a modified receptor or a DREADD using the methods described herein and exposing it to varying concentrations of a compound to be tested for modified receptor or DREADD binding. Modified receptor or DREADD binding can be detected exposure to the test compound, but not in the presence of a control compound that does not bind the modified receptor or DREADD and/or does not induce cellular activation.

In some aspects, the ligand can be clozapine-N-oxide (CNO), which is a metabolite of clozapine. In some aspects, the ligand can be perlapine, which binds to hM3Dq. Since the binding sites of hM3Dq and hM4Di are highly similar, it can likewise be expected to bind hM4Di.

Agonists and Dosage. The term “treatment” as used herein in the context of treating a disease or disorder, can relate generally to treatment and therapy of a human subject or patient, in which some desired therapeutic effect is achieved, for example, the inhibition of the progress of the disease or disorder, and can include a reduction in the rate of progress, a halt in the rate of progress, regression of the disease or disorder, amelioration of the disease or disorder, and cure of the disease or disorder. Treatment as a prophylactic measure (i.e., prophylaxis, prevention) is also included.

In some aspects, an exogenous ligand can be delivered in a therapeutically-effective amount. In some aspects, the nucleic acid constructs can be delivered in a therapeutically-effective amount.

The term “therapeutically-effective amount” as used herein, refers to the amount of the nucleic acid construct or exogenous ligand that is effective for producing some desired therapeutic effect, commensurate with a reasonable benefit/risk ratio, when administered in accordance with a desired treatment regimen.

Similarly, the term “prophylactically effective amount,” as used herein refers to the amount of the nucleic acid construct or exogenous ligand that is effective for producing some desired prophylactic effect, commensurate with a reasonable benefit/risk ratio, when administered in accordance with a desired treatment regimen. “Prophylaxis” as used herein refers to a measure which is administered in advance of detection of a symptomatic condition, disease or disorder with the aim of preserving health by helping to delay, mitigate or avoid that particular condition, disease or disorder.

While it may possible for the exogenous ligand to be used (e.g., administered) alone, it is often preferable to present it as a composition or formulation e.g. with a pharmaceutically acceptable carrier or diluent.

In some aspects, the ligand can be clozapine-N-oxide (CNO), which is a metabolite of clozapine. In some aspects, the CNO can be administered via parenteral administration. In some aspects, the CNO can be administered via oral administration. In some aspects, the dosage of CNO administered can be between 0.1 mg/kg and 20 mg/kg. In some aspects, the dosage of CNO administered can be between 1 mg/kg and 5 mg/kg.

The term “pharmaceutically acceptable,” as used herein, relates to compounds, ingredients, materials, compositions, dosage forms, etc., which are, within the scope of sound medical judgment, suitable for use in contact with the tissues of the subject (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some aspects, the nucleic acid constructs can be formulated to be delivered to cells and organisms in vitro and in vivo in a manner that allows the nucleic acid constructs to carry out their desired biological function. Delivery of nucleic acid constructs can be achieved through the use of viral vectors. In some aspects, delivery of nucleic acid constructs can be achieved through without the use of viral vectors. In some aspects, the nucleic acid constructs disclosed herein can be transfected into cells complexed with cationic lipids as well as a variety of other molecules.

In some aspects, the composition can be a pharmaceutical composition (e.g., formulation, preparation, medicament) comprising, or consisting essentially of, or consisting of as a sole active ingredient, a ligand as described herein, and a pharmaceutically acceptable carrier, diluent, or excipient.

In some aspects, the disclosed methods or compositions can be combined with other therapies, whether symptomatic or disease modifying.

The term “treatment” includes combination treatments and therapies, in which two or more treatments or therapies are combined, for example, sequentially or simultaneously. For example it may be beneficial to combine treatment with a compound as described herein with one or more other (e.g., 1, 2, 3, 4) agents or therapies. Appropriate examples of co-therapeutics are known to those skilled in the art based one the disclosure herein. Typically the co-therapeutic can be any known in the art which it is believed may give therapeutic effect in treating the diseases or disorders described herein, subject to the diagnosis of the individual being treated. The particular combination would be at the discretion of the physician who would also select dosages using his/her common general knowledge and dosing regimens known to a skilled practitioner.

The agents (e.g., a disclosed nucleic acid construct and exogenous ligand (or other therapeutic agent depending on the disorder or disease to be treated), plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

Disclosed herein are methods of treating a patient. In some aspects, the patient can be in need of any of the nucleic acid constructs disclosed herein.

### Pharmaceutical Compositions

As disclosed herein, are pharmaceutical compositions, comprising the nucleic acid constructs disclosed herein and a pharmaceutical acceptable carrier described herein. The compositions of the present disclosure also contain a therapeutically effective amount of a nucleic acid construct as described herein. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the non-immunogenic bioconjugate. Thus, compositions can be prepared for parenteral administration that includes nucleic acid constructs dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of condition disorder or disease.

The nucleic acid constructs described herein can be administered to the subject (e.g., a human patient) in an amount sufficient to delay, reduce, or preferably prevent the onset of clinical disease. Accordingly, in some aspects, the patient can be a human patient. In therapeutic applications, compositions are administered to a subject (e.g., a human patient) already with or diagnosed with a condition, disorder or disease in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of the nucleic acid constructs described herein can be an amount that achieves a cure, but that outcome is only one among several that can be achieved. One or more of the symptoms can be less severe. Recovery can be accelerated in an individual who has been treated.

The therapeutically effective amount of the nucleic acid constructs (and any additional therapeutic agent(s) to be combined with the nucleic acid constructs) described herein and used in the methods as disclosed herein applied to mammals (e.g., humans) can be determined by one of ordinary skill in the art with consideration of individual differences in age, weight, and other general conditions (as mentioned above).

Any of the compositions disclosed herein including the nucleic acid constructs described herein can be formulated for administration by any of a variety of routes of administration.

### EXAMPLES

### Example 1: Cell Type-Specific Alternative Splicing can be Targeted Using a Two-Color Fluorescent Reporter

Alternative splicing can be detected using next-generation sequencing. Alternative splicing of pre-mRNA generates extensive transcriptomic and proteomic diversity across most cell types. With the advent of next-generation RNA-Seq data, researchers can rapidly profile the entire transcriptome. The utility of RNA-Seq data has made it one of the most common experimental data types stored in public data archives. However, this data is often difficult to analyze effectively, especially with regard to analysis of RNA splicing. To reduce the computational barriers to entry for the general researcher, a resource (ASCOT, http://ascot.cs.jhu.edu/) that summarizes alternative splicing events across tens of thousands of publicly available RNA-Seq datasets (Ling J P, et al. (2018) ASCOT identifies key regulators of neuronal subtype-specific splicing. bioRxiv. doi:10.1101/501882) was developed. ASCOT does not rely on previously recorded splicing events and can therefore detect novel and unannotated splicing. Compared to commonly used gene annotations such as GENCODE or RefSeq, approximately 25% of splicing events found in ASCOT are novel (Ling J P, et al. (2018) ASCOT identifies key regulators of neuronal subtype-specific splicing. bioRxiv. doi:10.1101/501882). With this resource, thousands of alternative exons that are selectively used across the nervous system compared to the rest of the body were identified (FIG. 1). Furthermore, some of these unannotated splicing events are highly cell type-specific (FIG. 2).

Described herein are compositions and methods that use cell type-specific splicing to drive selective expression of reporter and effector constructs. To achieve this, highly cell type-specific alternative exons were identified, and mutations that would lead to a reading frame shift if spliced into messenger RNA (mRNA) were introduced. Next, a modified two-color fluorescent reporter was used to validate this approach (FIG. 3) (Orengo J P, Bundman D, Cooper T A (2006) Nucleic Acids Res; and Zheng S (2017). Methods Mol Biol 1648:221-233). In this construct, an ATG start codon is placed upstream of an intron containing a cell type-specific exon. Following this intron is the coding sequences for mCherry and green fluorescent protein with a nuclear localization signal (NLS-GFP), which are offset by a single base pair frameshift. In most cells, normal splicing will produce mRNA that encode mCherry. In the targeted cell type, however, the alternatively spliced mRNA will produce a coding sequence that reads through mCherry into NLS-GFP.

Validated plasmid-based SLED dual fluorescent reporter constructs that specifically target muscles, neurons, and rod photoreceptors. As a proof of concept for this approach, several constructs were generated that express NLS-GFP in a specific cell type (FIG. 4). Muscles, neurons, and photoreceptors have many cell type-specific exons that are suitable for the splicing-linked expression design (SLED) strategy (Ling J P, et al. (2018) ASCOT identifies key regulators of neuronal subtype-specific splicing. bioRxiv. doi:10.1101/501882). Finally, muscle-specific expression in mixed cultures of human fibroblasts and myotubes using SLED constructs derived from a muscle-specific exon of Spag9 (FIGS. 4A-D) was observed. In vitro transfection was used to validate appropriate expression of the SLED construct derived from a pan-neuronal-specific exon of Pls3 (FIGS. 4E-H) and an excitatory neuron-specific exon of Synrg in mixed primary cultures from rat hippocampus (FIGS. 4I-L). Finally, using in vivo electroporation of mouse retina at P0, it was shown that plasmid-based SLED constructs derived from a photoreceptor-specific splice form of Atp1b2 show rod-specific expression at P14 (FIGS. 4M-Q). A total of 17 such plasmid-based SLED constructs were tested, and highly cell type-specific expression in 14 (82%) constructs was found, suggesting that the method used for identifying SLED introns is highly robust.

### Example 2: Development of AAV- and Lentivirus-Based SLED Reporter Constructs that Selectively Target Specific Cell Types in Mouse Nervous System

Constructs will be generated that selectively target primary sensory neurons in the auditory, somatosensory and olfactory system, as well as primary motor neurons. In parallel, both excitatory and inhibitory neurons, astrocytes and oligodendrocytes in cerebral cortex will be targeted. Finally, using an integrated analysis of full-length single cell RNA-Seq data from multiple studies, SLED-based constructs will be combined with cell type-specific promoters to selectively target highly specific subtypes of excitatory and inhibitory cortical neurons.

Cell identity in the nervous system can be defined through a hierarchical taxonomy, with each level reflected in common patterns of gene expression (Zeng H, Sanes J R (2017) Nat Rev Neurosci 18(9):530-546). At the top of this taxonomic hierarchy sit broad categories such as neurons and glia; just below sit excitatory, inhibitory and primary sensory neurons; below them sit categories such as layer-specific identity of pyramidal neurons and major subtypes of cortical interneurons, and so on down to the level of individual cell types (FIG. 7). Using ASCOT analysis of publicly available bulk RNA-Seq datasets, it was found that alternative splicing patterns show a broadly similar organization. Many genes show neuron or glia-specific patterns of splicing. Smaller numbers of genes show splicing patterns specific to either primary motor and sensory neurons—including photoreceptors, olfactory sensory neurons, somatosensory neurons of the dorsal root ganglion, and cochlea hair cells—as well as excitatory and inhibitory neurons, astrocytes and oligodendrocyte-specific splicing patterns. Below this level, very few splicing patterns are absolutely cell type-specific, but many are cell-type specific within the taxonomic level in question. The recent availability of high-quality full-length single cell RNA-Seq datasets from both mouse and human cortex (Hodge R D, et al. (2019) Nature. doi:10.1038/s41586-019-1506-7), as well as Ribotrap RNA-Seq data from major subtypes of cortical interneurons (Furlanis E, et al. (2019) Nat Neurosci. doi:10.1038/s41593-019-0465-5), now makes it possible to identify splicing patterns that are specific to individual subsets of cortical cell types. When combined with cell type-specific promoter constructs, SLED constructs based on these can theoretically direct absolutely cell type-specific patterns of reporter and effector gene expression.

This dataset can be used to design and test AAV and lentiviral dual fluorescent reporter SLED constructs that will selectively target reporter gene expression to specific cell types of the nervous system. First, primary sensory and motor neurons will be targeted, which present the largest number of highly cell type-specific alternative splicing events, and which show particularly high levels of cell type-specific splicing. Next constructs selectively expressing in higher taxonomic levels of cell identity in cortex (Levels 1 and 2 in FIG. 7), targeting excitatory and inhibitory neurons, as well as astrocytes and oligodendrocytes will be generated. Finally, cell type-specific promoter and SLED sequences will be combined to generate constructs that selectively target more specific subtypes of cortical excitatory and inhibitory neurons (Levels 3 and 4 in FIG. 7).

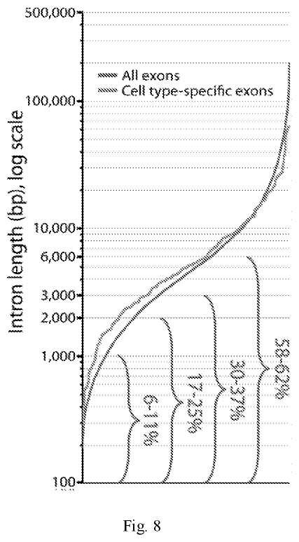

Proposed experiments: Identification of AAV and lentiviral SLED constructs that express selectively in primary sensory and motor neurons. Using the comprehensive alternative splicing ASCOT database (Ling J P, et al. (2018) ASCOT identifies key regulators of neuronal subtype-specific splicing. bioRxiv. doi:10.1101/501882), candidate splicing events will be identified that are suitable for SLED design. Special emphasis will be taken to ensure maximal likelihood of success for downstream applications regarding AAV delivery and tool development. The four ranked main selection criteria for selecting sequences for use in SLED constructs. In order, these are [A] cell type percent spliced-in (PSI) specificity, [B] evolutionary conservation, [C] intron length, and [D] cross validation with different RNA-Seq datasets in the public archive. For the first criterion, a splicing event must be above 30 percent spliced in (PSI) in the cell type of interest and near 0 PSI in other non-target cell types that may express a SLED construct. Second, alternative splicing events should be evolutionarily conserved across vertebrates. Conservation can be determined based on phyloP scores (Cooper G M, et al. (2005) Genome Res 15(7):901-913) of the intronic sequence itself, as well as validated using RNA-Seq splicing analysis from multiple species when such datasets are available. Third, alternative splicing events with shorter intron lengths will be prioritized given the maximal packing capacity of ˜5 kb for AAVs. Thus, introns<3 kb will be suitable for AAV vector development. It is estimated that ˜20-30% of cell type-specific exons meet this criterion, while ˜60% will be suitable for design of lentiviral constructs (FIG. 8). Finally, alternative exons that are identified in multiple independent RNA-Seq datasets will be prioritized. For example, the cochlear hair cell-specific exon in Sptan1 (FIG. 2) was validated in both FACS isolated bulk sequencing data (Cai T, et al. (2015) J Neurosci 35(14):5870-5883.) as well as a separate full length single cell RNA-Seq study (Burns J C, et al. (2015) Nat Commun 6:8557). As additional RNA-Seq data becomes available, it will be incorporated into ASCOT to improve and refine construct design.

Table 2 is a list of candidate introns to use for SLED. The following cell types will be targeted: olfactory sensory neurons (OSNs), cochlea hair cells, somatosensory neurons and primary motor neurons. These are chosen since strongly cell type-specific expression constructs for these cell types are lacking. Three constructs targeting each of these cell types will be generated. The ubiquitous Ef1a promoter sequence will be used to drive transcription of these reporters (Sohal V S, et al. (2009) Nature 459(7247):698-702). Exon/intron constructs that are <3 kb will be cloned into dual reporter AAV vectors, with site-directed mutations incorporated into the coding sequence of the distal exon as needed to preserve reading frame. Alternative exon/intron sequences that are >3 kb will be tested in dual fluorescent lentiviral vectors, and processed similarly (FIG. 3). The serotypes for AAV stocks will be selected based on published reporters (Table 3). Viral stocks will then be delivered to the target organs using intranasal (OSN), round window (cochlea hair cells), and intrathecal (somatosensory and motor neurons) injection (Williams C L, et al. (2017) Mol Ther 25(4):904-916; Chien W W, et al. (2015) Laryngoscope 125(11):2557-2564; Wang H, et al. (2014) Hum Mol Genet 23(3):668-681; and Yu H, et al. (2016) Methods Mol Biol 1382:251-261).

Three sequential criteria will be used for measuring the efficiency and specificity of SLED constructs. First, 14-28 days following infection, infected tissue will be harvested and dissociated, and GFP and mCherry-positive cell populations will be separated via FACS. It is expected that several distinct populations of cells will be visualized: mCherry-positive cells, which do not express the cell type-specific exon in question; GFP+ cells, in which the alternative exon is being spliced in a cell type-specific manner; and a variable level of mCherry+/GFP+ cells, in which both splicing patterns are observed, the numbers of which will depend on the level of specificity of the alternative exon in question. SLED reporters in which 70% of signal is found in GFP-only cells will be processed for further analysis. Confirmation of successful infection of the target cell type will be determined by qRT-PCR analysis using well-established cell type-specific markers listed in Table 4.

SLED reporters that pass this first specificity test will then be processed for immunohistochemistry and/or single-molecule fISH analysis, using cell type-specific markers listed in Table 3. The fraction of GFP-positive cells that stain with the marker in question will be quantified using standard approaches (de Melo J, et al. (2018) Development 145(9)), and constructs in which >70% of GFP-positive cells express the marker in question will be processed further.

The gene expression profile of SLED reporters that pass these criteria will be comprehensively profiled using MULTI-Seq. GFP-positive cells will be isolated by FACS as described herein, scRNA-Seq libraries will be generated using MULTI-Seq, as described. Cell type-specific expression will be determined based on enrichment of cell type-specific gene expression profiles determined by analysis of publicly available scRNA-Seq datasets from the target tissues. SLED constructs that show >80% specificity based on these criteria will be used for further experiments described herein.

AAV and lentiviral SLED constructs that selectively target excitatory and inhibitory cortical and hippocampal neurons, as well as cortical astrocytes and oligodendrocytes will be identified. Three SLED constructs will be tested that target excitatory neurons, inhibitory neurons, astrocytes and oligodendrocytes, respectively, for a total of 12. To improve the specificity of the neuronal SLED constructs, however, the pan-neuronal hSyn promoter will be used to drive expression, but the EF1a promoter will be used to drive expression of glial-specific constructs (Sohal V S, et al. (2009) Nature 459(7247):698-702). Viruses will be delivered to the visual cortex and hippocampus via stereotactic injection (Lowery R L, Majewska A K (2010) J Vis Exp (45). doi:10.3791/2140; and Keiser M S, et al. (2018) Curr Protoc Mouse Biol 8(4):e57). Specificity will be analyzed using FACS, histology, and MULTI-Seq, as described above.

Lastly, AAV and lentiviral SLED constructs that target more highly specific subtypes of excitatory and inhibitory cortical neurons will be designed. Specifically, these include layers 2, 3, 4 and 5-specific excitatory pyramidal neurons; constructs that express broadly in either Pva1b, Sst or Vip-positive interneurons; and constructs which target specific subtypes of Sst and Vip-positive interneurons. A total of 20 SLED constructs that target a broad range of these neuronal subtypes will be tested. The hSyn promoter will be used to drive expression of excitatory neuron-specific SLED constructs, while inhibitory neuron-specific constructs will be driven by either the hSyn or CamKII promoters for excitatory neurons (Kügler S, et al. (2003) Gene Therapy 10(4):337-347; and Watakabe A, et al. (2015) Neurosci Res 93:144-157), or the Dlx enhancer-derived sequence for inhibitory neurons (Dimidschstein J, et al. (2016) Nat Neurosci 19(12):1743-1749), depending on the specificity of the splicing pattern in question. Injections and specificity analysis will be conducted as described herein.

In cases where successful lentiviral SLED constructs are identified, a series of deletion analysis, removing evolutionarily non-conserved intronic sequences to reduce total length to <3 kb will be conducted. These will be performed on lentiviral constructs with inserts<4.5 kb, which is expected to comprise roughly 50% of the passing constructs. A maximum of 3 miniaturized constructs will be tested for each passing construct. Miniaturized lentiviral inserts that show faithful cell type-specific expression will then be retested in AAV as described herein.

TABLE 2
Selected list of SLED constructs to be generated.

| | Genomic locus | Size | Targeted | PSI/ | Ave | | | PhyloP |
|---|---|---|---|---|---|---|---|---|
| Gene | (mm 10) | (bp) | Cell | target | PSI | Promoter | Ref | conservation? |
| Zfc3h1 | chr10: 115427791- | 1070 | OSN | 30.5 | 0.2 | Ubiquitous | Saraiva LR, et | Yes |
| | 115428860 | | | | | | al. (2015) | |
| | | | | | | | Sci Rep | |
| | | | | | | | 5: 18178. | |
| Sptan1 | chr2: 29980203- | 2957 | Cochlea | 85.1 | 0.1 | Ubiquitous | Cai T, et | Yes |
| | 29983159 | | hair cells | | | | al. (2015) | |
| | | | | | | | J Neurosci | |
| | | | | | | | 35(14): | |
| | | | | | | | 5870-5883 | |
| Ptprf | chr4: 118220609- | 2617 | DRG | 79.5 | 0.4 | Ubiquitous | Tedeschi A, et | Yes |
| | 118223225 | | neurons | | | | al. (2016) | |
| | | | | | | | Neuron | |
| | | | | | | | 92(2): | |
| | | | | | | | 419-434 | |
| Itga6 | chr2: 71822570- | 2939 | Motor | 77.6 | 0.4 | Ubiquitous | Amin ND, et | Yes |
| | 71825508 | | neurons | | | | al. (2015) | |
| | | | | | | | Science | |
| | | | | | | | 350(6267): | |
| | | | | | | | 1525-1529 | |
| Sestd1 | chr2: 77192581- | 4242 | Astrocytes | 24.0 | 1.2 | Ubiquitous | Zhang Y, et | Yes |
| | 77196822 | | | | | | al. (2014) | |
| | | | | | | | J Neurosci | |
| | | | | | | | 34(36): | |
| | | | | | | | 11929-11947 | |
| Phldb1 | chr9: 44687699- | 918 | Oligodendrocytes | 80.8 | 0.2 | Ubiquitous | Zhang Y, et | Yes |
| | 44688616 | | | | | | al. (2014) | |
| | | | | | | | J Neurosci | |
| | | | | | | | 34(36): | |
| | | | | | | | 11929-11947 | |
| Camk2a | chr18: 60963964- | 6030 | GABAergic | 58.1 | 1.4 | hSyn/mDlx | Tasic B, et | Yes |
| | 60969993 | | neurons | | | | al. (2016) | |
| | | | | | | | Nat Neurosci | |
| | | | | | | | 19(2): | |
| | | | | | | | 335-346 | |
| Twf1 | chr15: 94582876- | 1505 | Pvalb+ | 22.7 | 0.3 | mDlx | Tasic B, et | Yes |
| | 94584380 | | neurons | | | | al. (2016) | |
| | | | | | | | Nat Neurosci | |
| | | | | | | | 19(2): | |
| | | | | | | | 335-346 | |
| Cask | chrX: 13526082- | 7258 | Vip+ | 29.0 | 1.6 | mDlx | Tasic B, et | Yes |
| | 13533339 | | neurons | | | | al. (2016) | |
| | | | | | | | Nat Neurosci | |
| | | | | | | | 19(2): | |
| | | | | | | | 335-346 | |
| Stx17 | chr4: 48140486- | 18325 | Sst+/Chodl+ | 34.1 | 0.0 | mDlx | Tasic B, et | Yes |
| | 48158810 | | neurons | | | | al. (2016) | |
| | | | | | | | Nat Neurosci | |
| | | | | | | | 19(2): | |
| | | | | | | | 335-346 | |
| Nsd1 | chr13: 55276642- | 890 | Vip+/Sncg+ | 51.4 | 0.0 | mDlx | Tasic B, et | Yes |
| | 55277531 | | Neurons | | | | al. (2016) | |
| | | | | | | | Nat Neurosci | |
| | | | | | | | 19(2): | |
| | | | | | | | 335-346 | |
| Zfp512 | chr5: 31472962- | 491 | Sst+/Th+ | 89.1 | 0.0 | mDlx | Tasic B, et | No |
| | 31473452 | | neurons | | | | al. (2016) | |
| | | | | | | | Nat Neurosci | |
| | | | | | | | 19(2): | |
| | | | | | | | 335-346 | |
| Msl3 | chrX: 168654969- | 1994 | Pvalb+/Cpne5+ | 37.5 | 0.0 | mDlx | Tasic B, et | No |
| | 168656962 | | neurons | | | | al. (2016) | |
| | | | | | | | Nat Neurosci | |
| | | | | | | | 19(2): | |
| | | | | | | | 335-346 | |
| Dock9 | chr14: 121576020- | 2135 | Layer 2 | 44.9 | 0.6 | CamkII | Tasic B, et | Yes |
| | 121578154 | | Ngb+ | | | | al. (2016) | |
| | | | neurons | | | | Nat Neurosci | |
| | | | | | | | 19(2): | |
| | | | | | | | 335-346 | |
| Sntg1 | chr1: 8607153- | 17626 | Layer 2/3 | 32.1 | 0.4 | CamkII | Tasic B, et | No |
| | 8624778 | | Ptgs2+ | | | | al. (2016) | |
| | | | neurons | | | | Nat Neurosci | |
| | | | | | | | 19(2): | |
| | | | | | | | 335-346 | |
| Parp1 | chr1: 180580636- | 2122 | Layer 4 | 32.1 | 0.0 | CamkII | Tasic B, et | No |
| | 180582757 | | Scnn1a+ | | | | al. (2016) | |
| | | | neurons | | | | Nat Neurosci | |
| | | | | | | | 19(2): | |
| | | | | | | | 335-346 | |
| Fgfr1op | chr17: 8186582- | 4816 | Layer 4 | 43.3 | 0.0 | CamkII | Tasic B, et | No |
| | 8191397 | | Arf5+ | | | | al. (2016) | |
| | | | neurons | | | | Nat Neurosci | |
| | | | | | | | 19(2): | |
| | | | | | | | 335-346 | |
| Nbas | chr12: 13289981- | 10112 | Layer 5a | 44.5 | 0.0 | CamkII | Tasic B, et | Yes |
| | 13300092 | | Hsd11b1+ | | | | al. (2016) | |
| | | | neurons | | | | Nat Neurosci | |
| | | | | | | | 19(2): | |
| | | | | | | | 335-346 | |
| Btbd1 | chr7: 81797084- | 3874 | Layer 6a | 91.1 | 0.5 | CamkII | Tasic B, et | Yes |
| | 81800957 | | Syt17+ | | | | al. (2016) | |
| | | | neurons | | | | Nat Neurosci | |
| | | | | | | | 19(2): | |
| | | | | | | | 335-346 | |

AAV serotypes for optimal targeting of each cell type are known in most cases. Each cell types tested has also been previously isolated via FACS, and each has been previously profiled via scRNA-Seq (Hodge R D, et al. (2019) Nature. doi:10.1038/s41586-019-1506-7; Dang P, et el. (2018) PLoS Genet 14(1):e1007164; Li Y, et al. (2018) Sci Data 5:180199; Li C, et al (2018) Neurosci Bull 34(1):200-207; and Rosenberg A B, et al. (2018) Science 360(6385):176-182).

TABLE 3
AAV serotypes to be used for targeting specific cell types.

| Cell type | Rodent | Ferret | Human | References |
|---|---|---|---|---|
| Excitatory | AAV-PHP.eB, | AAV2/1 | AAV7m8, AAV6 | (Wilson D E, et al. (2017) |
| neurons | AAV9, AAV5 | | | Neuron 93(5): 1058-1065.e4; |
| | | | | Watakabe A, et al. (2015) |
| | | | | Neurosci Res 93: 144-157; |
| | | | | Gray S J, et al. (2011) |
| | | | | 19(6): 1058-1069; |
| | | | | Chan K Y, et al. (2017) Nat |
| | | | | Neurosci 20(8): 1172-1179; |
| | | | | and Duong T T, et al. (2019) |
| | | | | Stem Cells International |
| | | | | 2019: 1-11) |
| Inhibitory | AAV-PHP.eB, | AAV2/1 | AAV7m8, AAV6 | (Wilson D E, et al. (2017) |
| neurons | AAV9, AAV5 | | | Neuron 93(5): 1058-1065.e4; |
| | | | | Watakabe A, et al. (2015) |
| | | | | Neurosci Res 93: 144-157; |
| | | | | Gray S J, et al. (2011) |
| | | | | 19(6): 1058-1069; |
| | | | | Chan K Y, et al. (2017) Nat |
| | | | | Neurosci 20(8): 1172-1179; |
| | | | | and Duong T T, et al. (2019) |
| | | | | Stem Cells International |
| | | | | 2019: 1-11) |
| Motor | AAV-PHP.eB, | NA | AAV9 | (Chan K Y, et al. (2017) Nat |
| neurons | AAV9, | | | Neurosci 20(8): 1172-1179; |
| | | | | Foust K D, et al. (2009) Nat |
| | | | | Biotechnol 27(1): 59-65; and |
| | | | | Mendell J R, et al. (2017) N |
| | | | | Engl J Med 377(18): 1713- |
| | | | | 1722) |
| Somatosensory | AAV-PHP.S, | NA | AAV2 | (Chan K Y, et al. (2017) Nat |
| neurons | AAV9, AAV5 | | | Neurosci 20(8): 1172-1179; |
| | | | | Mason M R J, et al. (2010) |
| | | | | Comparison of AAV |
| | | | | Serotypes for Gene Delivery |
| | | | | to Dorsal Root Ganglion |
| | | | | Neurons. Mol Ther |
| | | | | 18(4): 715; Hirai T, et al. |
| | | | | (2014) Molecular Therapy |
| | | | | 22(2): 409-419; and Fleming |
| | | | | J, et al. (2001) Hum Gene |
| | | | | Ther 12(1): 77-86) |
| Oligodendrocytes | AAV-PHP.eB, | AAV2/1 | AAV7m8, AAV6 | (Wilson D E, et al. (2017) |
| | AAV9, AAV8 | | | Neuron 93(5): 1058-1065.e4; |
| | | | | Chan K Y, et al. (2017) Nat |
| | | | | Neurosci 20(8): 1172-1179; |
| | | | | and Duong TT, et al. (2019) |
| | | | | Stem Cells International |
| | | | | 2019: 1-11; and Aschauer |
| | | | | D F, et al. (2013). PLoS ONE |
| | | | | 8(9): e76310) |
| Astrocytes | AAV-PHP.eB, | AAV2/1 | AAV7m8, AAV6 | (Wilson D E, et al. (2017) |
| | AAV9, AAV5 | | | Neuron 93(5): 1058-1065.e4; |
| | | | | Watakabe A, et al. (2015) |
| | | | | Neurosci Res 93: 144-157; |
| | | | | Gray S J, et al. (2011) |
| | | | | 19(6): 1058-1069; |
| | | | | Chan K Y, et al. (2017) Nat |
| | | | | Neurosci 20(8): 1172-1179; |
| | | | | and Duong T T, et al. (2019) |
| | | | | Stem Cells International |
| | | | | 2019: 1-11) |
| Cochlea | AAV-DJ, | NA | NA | (Kim M-A, et al. (2019) Mol |
| hair cells | AAV2/2 | | | Ther Methods Clin Dev |
| | | | | 13: 197-204; and Gu X, et al. |
| | | | | (2019) Front Cell Neurosci |
| | | | | 13: 8) |
| OSNs | AAV9, AAV12 | NA | NA | (Williams C L, et al. (2017) |
| | | | | Mol Ther 25(4): 904-916; and |
| | | | | Quinn K, et al. (2011) Mol |
| | | | | Ther 19(11): 1990-1998) |

About <20% of the candidate sequences are short enough to be packaged efficiently into AAV vectors, although >70% are predicted to package efficiently into lentivirus. It is predicted that in many cases, by selectively deleting blocks of non-conserved intronic sequence, and avoiding removing sequences that are known cis-regulatory modulators of splicing (Rosenberg A B, et al. (2015) Cell 163(3):698-711), that we may be able to modify these to generate miniaturized SLED constructs which both fit efficiently in AAV vectors and express correctly. This general approach has been used to identify minimal intronic sequences required for regulation of alternative splicing.

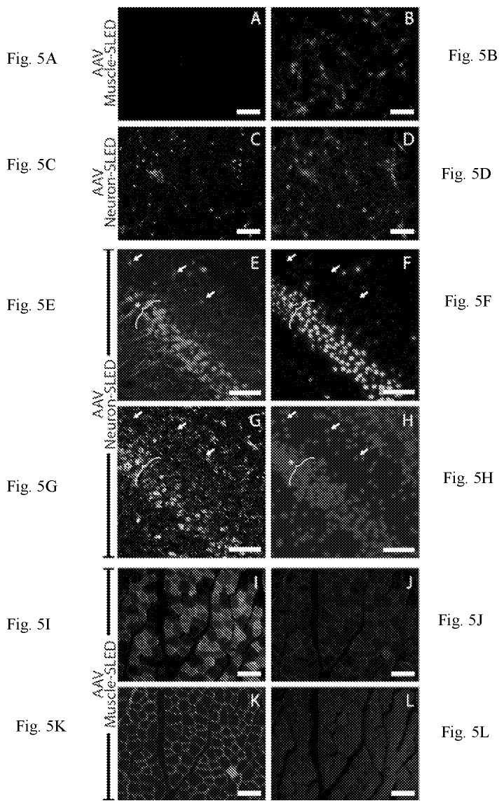

Validation of AAV-based SLED constructs. Having validated several constructs in multiple cell types and species, it was tested whether SLED constructs validated in plasmid vectors retained specificity when expressed in AAV vectors. The specificity of the muscle-specific Spag9-derived and pan-neuronal-specific P/s3-derived SLED constructs using primary cultures from E17 mouse forebrain (FIG. 5A-D) were first tested. No GFP expression was detected from the muscle-specific SLED AAV construct (FIG. 5A), but robust expression of GFP in most cells with the pan-neuronal SLED construct (FIG. 5C) was observed. A robust mCherry expression from both vectors was also observed, indicating efficient infection (FIG. 5B, D).

Next, the selectivity of both vectors were tested in vivo. Fourteen days following stereotaxic injection into the adult mouse hippocampus (FIGS. 5-E-H), robust NLS-GFP expression was observed from the pan-neuronal SLED vector in CA1 and in a small number of nearby interneurons (FIG. 5F), as determined by NeuN expression (FIG. 5G). mCherry-positive, NeuN-negative cells that likely represent glia are also visualized, which lack NLS-GFP expression, indicating that NLS-GFP is broadly but selectively expressed in neurons. Fourteen days following injection into adult limb muscle, the muscle-specific AAV SLED construct showed strong GFP signal in a subset of WGA-positive muscle fibers (FIGS. 5I-L). GFP signal is less clearly nuclear than expected, possibly due to the multinucleated nature of mature muscle fibers. The striking contrast between the robust GFP expression seen in muscle, and the absence of any detectable GFP expression in neurons, confirms the muscle-specificity of this AAV-based SLED construct.

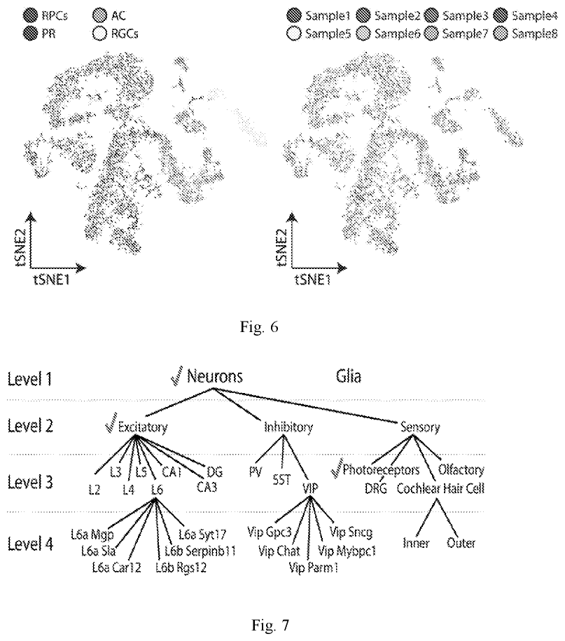

MULTI-Seq analysis allows inexpensive analysis of cell type identity. Droplet-based ScRNA-Seq analysis is the most rapid and comprehensive means of profiling cell types in a sample, but remains expensive. The use of MULTI-Seq, which incorporates sequence-based barcodes into the cells in a sample using affinity reagents (McGinnis C S, et al. (2019) Nat Methods 16(7):619-626), offers a means to combine multiple large numbers of samples into a single library preparation, reducing costs substantially. MULTI-Seq was used to simultaneously profile 8 samples of mouse retina which were explanted at E18, and shortly thereafter profiled in a single sequencing reaction. It was found that the 8 samples showed an approximately equal fraction of the major retinal cell types present in the sample (FIG. 6).

### Example 3: Test Selectivity of SLED Constructs Across Multiple Mammalian Species

Constructs that show highly cell type-specific expression in mouse will be tested in rat, ferret, and ES/iPS-derived human cells to determine if these cell type-specific expression patterns are retained across species.

Transgenic mouse models have revolutionized the understanding of cell types and circuits in the nervous system. However, transgenic approaches in other species require extensive resources and are often limited in scope. To directly address this challenge, SLED-based AAVs will be used as a delivery platform for targeting cell types across multiple mammalian species used for neuroscience research. Such tools would be valuable resources for the research community and expand the potential of using other model organisms for studying neural circuitry.

The alternative splicing events used to drive control expression of SLED constructs show varying levels of evolutionary conservation. This is particularly high in the case of primary sensory neurons. For instance, nearly half of the rod photoreceptor-specific alternative exons are conserved in RNA-Seq samples from mouse and humans, for instance, and over 70% of the rod-specific alternative exons that show strong primary sequence conservation also show retina-specific splicing in both mouse and human (Ling J P, et al. (2018) ASCOT identifies key regulators of neuronal subtype-specific splicing. bioRxiv. doi:10.1101/501882). Although there are far fewer RNA-Seq datasets available from other mammalian species than for mouse and human, this suggests that alternative exons whose primary sequence is conserved also have a high probability of showing conserved splicing patterns. Even a large fraction of exons specific to subtypes of cortical neurons show strong evolutionary conservation (Table 2), and would thus be predicted to have a high probability of showing selective expression in multiple mammalian species.

Studies will be conducted to determine whether SLED constructs that are validated in mouse as described herein, and that show evolutionarily conservation of alternative exon sequence, show selective expression in target cells in rat, ferret and human. In rat, constructs targeting the cell types tested in mouse will be tested. In ferret, cortical cell types will be tested. Also test constructs will be used targeting somatosensory and motor neurons, as well as cortical neurons and glia, using human ES/iPS-derived cells and organoids.

Proposed experiments: The specificity of SLED viral constructs that pass validation in mice will be tested in rats, mice and human cells, using the same approach described herein. Those constructs for which there is either clear evidence for evolutionary conservation of cell type-specific splicing, or the primary sequence of the alternative exon in question, will be used for these studies. A maximum of 15 SLED constructs will be tested. The same workflow for work carried out in rat will be used for mice, using AAVs of the same serotype where applicable, with 7 week old animals used for analysis.

SLED constructs that target cortical cells in both show appropriate cell type-specific expression in both mouse and rat will then be tested in ferrets. A maximum of 11 such constructs will be tested. A maximum of two constructs will be tested in each animal, with a separate construct delivered by stereotactic injection into each cortical hemisphere performed between postnatal days 27 and 29 (Wilson D E, et al. (2017) Neuron 93(5):1058-1065.e4). FACS analysis of transduced cells will be performed as described herein, while histological analysis of gene expression will be performed using custom-designed smfISH RNAScope probes that target cell type-specific markers listed in Table 3 (Johnson M B, et al. (2018) Nature 556(7701):370-375). 10× Chromium-based scRNA-Seq will be used to generate a reference cellular expression atlas from postnatal day 45 ferret cortex (Clark B S, et al. (2019) Neuron 102(6):1111-1126.e5; and Yoo S, et al. (2019) Front Neurosci 13:240), and will be used to analyze cell specificity of any constructs profiled using MULTI-Seq. AAV viral serotypes used for these studies are listed in Table 3.

Lastly, SLED construct specificity in human cells will be validated. Somatosensory and motor neuron-specific SLED constructs that validate in rat will be tested for their ability to selectively express in human ES cell-derived somatosensory and motor neurons maintained in culture. Somatosensory neurons will be generated from H9 human ES cells through a process of directed differentiation (Oh Y, et al. (2017) Nat Neurosci 20(9):1209-1212), transduced at 22-28 days in vitro, and cultured for 14 days. Somatosensory neuron-containing mixed cultures will be transduced with SLED reporter constructs at days in vitro, and profiled using FACS as described herein. Histological analysis will be performed with immunocytochemistry to HNK-1 (B3gat1) and Tfap2a (Oh Y, et al. (2017) Nat Neurosci 20(9):1209-1212). Motor neurons will be generated from iPS cells (d′Ydewalle C, et al. (2017) Neuron 93(1):66-79), with cultured infected with SLED reporter constructs at 15 days in vitro and analyzed 14 days later, and specificity characterized via FACS, immunocytochemistry, and MULTI-Seq as described herein. For each of these human cell types, up to 2 SLED constructs will be characterized.

Cortical cell type-specific SLED constructs that validate in ferret using human ES-derived cells will be tested, using both cortical organoids and organoid-derived cells transplanted into rats. A maximum of 5 SLED reporter targeting major subtypes of human cortical neurons and glia will be first tested using cortical organoids, which will be generated (Qian X, et al. (2016) Cell 165(5):1238-1254). After 30 days in vitro, these will be infected with SLED constructs, and processed 14 days later for FACS, immunostaining, and MULTI-Seq. If these express faithfully, the specificity of a maximum of 2 constructs will then be tested for using CFP-expressing hES cell-derived cortical progenitors grafted into neonatal rat (Yin X, et al. (2019) eNeuro 6(4). doi:10.1523/ENEUR0.0148-19.2019). SLED constructs will be injected into rat cortex at 7 weeks postnatal, and processed for FACS, immunostaining, and MULTI-Seq.

If useful SLED reagents are determined, then the development of reagents that selectively label many, if not most, of the cell types described herein will be generated. The high level of conservation of protein coding sequence, and the ready availability of custom smfISH reagents in the more evolutionarily distant ferret, should make identification of specific cell types relatively straightforward.

### Example 4: Develop SLED-Based Functional Tools to Selectively Monitor and Modulate Cell Activity

SLED-based reporter constructs that show highly cell type-specific expression patterns will be modified to express additional effector molecules. These will include but are not limited to calcium sensors (GCaMP7), channelrhodopsin-2 (ChR2), or Designer Receptors Exclusively Activated by Designer Drugs (DREADDs). These constructs will then be tested in vivo for their ability to monitor and modulate cellular activity.

SLED-based reagents, particularly when combined with existing promoters, will allow highly cell type-specific expression of a broad range of molecular tools useful for analyzing neural circuitry in multiple mammalian species.

Described herein are SLED-based reagents that can be used to deliver effector constructs that record and modulate neuronal activity and/or function to specific cell types in the nervous system. To this end, the SLED reporter vectors described herein can be adapted to express calcium sensors, channelrhodopsin, DREADDs, as well as any individual genes. The efficacy and specificity of these reagents will then be tested in vivo.

TABLE 4
Molecular markers used for validation of SLED constructs.

| Cell type | Antibody targets | Reference |
|---|---|---|
| Excitatory | Slc17a6, Slc17a7 | Tasic B, et al. (2016) Nat Neurosci |
| neurons | | 19(2): 335-346 |
| Inhibitory neurons | Gad1/Gad2, Sst, | Tasic B, et al. (2016) Nat Neurosci |
| | Pvalb, Vip | 19(2): 335-346 |
| Motor neurons | Isl1, Chat | Cho H-H, et al. (2014) PLoS Genet |
| | | 10(4): e1004280 |
| Somatosensory | Prph, Nefh, | Li C-L, et al. (2016) Cell Res |
| neurons | Tfap2a, B3gat1 | 26(8): 967 |
| Oligodendrocytes | Pdgfra, Mbp, Olig1 | Tasic B, et al. (2016) Nat Neurosci |
| | | 19(2): 335-346 |
| Astrocytes | Aldh1l1, Slc1a3 | Tasic B, et al. (2016) Nat Neurosci |
| | | 19(2): 335-346 |
| Cochlea hair cells | Atoh1, Myo7a | Li Y, et al. (2018) Sci Data 5: 180199 |
| OSNs | Omp, Adcy3 | Zhang Z, et al. (2017) Front Cell |
| | | Neurosci 11: 1 |

Proposed experiments: SLED-based dual fluorescent reporter constructs will be adapted to express the following constructs: GCaMP7 (Dana H, et al. (2019) Nature Methods 16(7):649-657), ChR2-eYFP, (Nagel G, et al. (2003) Proc Natl Acad Sci USA 100(24):13940-13945), and hM3Dq-mCherry Gq-coupled DREADDs (Armbruster B N, et al. (2007) Proc Natl Acad Sci USA 104(12):5163-5168). Additional overexpression or rescue constructs will be generated in line including but not limited to ciliary proteins (Talaga A K, et al. (2017) J Neurosci 37(23):5699-5710), G-protein coupled receptors (He S-Q, et al. (2018) Sci Signal 11(535)), or enzymes regulating phosphorylation or glycosylation of neurotransmitter receptors (Lagerlof O, et al. (2017) Proc Natl Acad Sci USA 114(7):1684-1689; and Hussain N K, et al. (2015) Proc Natl Acad Sci USA 112(43):E5883-90). A maximum of one construct that is specific to a given cell type and has passed validation will be modified in this manner. Each alternative exon will generate a stretch of N-terminal leader sequence that may potentially interfere with the expression or function of the effector protein when expressed, which poses a particular problem for membrane proteins containing signal peptides such as Gq DREADDs. To avoid this problem, a P2A self-cleaving peptide sequence will be inserted immediately 3′ of the alternative exon (Liu Z, et al. (2017) Scientific Reports 7(1). doi:10.1038/s41598-017-02460-2), inserting extra bases as needed to maintain the frame of translation. For each construct, this should then result in the production of a short leader peptide and the effector protein of interest. Specific expression of the effector construct of interest will be determined using intrinsic fluorescence and cell type-specific markers described herein. These constructs can then be used for functional analysis of cell types of interest.

The effector SLED constructs generated are either approximately the same size or shorter than the dual fluorescent reporter construct, and thus should package efficiently into AAV or lentiviral vectors. P2A sequences typically drive protein expression at comparable levels of expression for both N- and C-terminal proteins (Liu Z, et al. (2017) Scientific Reports 7(1). doi:10.1038/s41598-017-02460-2), and thus efficient and equivalent expression levels of the leader sequence and effector gene of interest is expected. A series of point mutants can also be generated in the upstream sequence and variants that both preserve cell type-specific splicing patterns reduce cytotoxicity can be identified.

For the experiments described herein, the following methods can be applied.

Statistical analysis: Two-way ANOVA analysis will be used to assess significance for the cell counting data. For MULTI-Seq analysis, analysis will be used to identify cell clusters, with non-parametric binomial analysis then used to each cluster to one another to identify differentially expressed genes, in combination with Benjamini-Hochberg correction to control for false discovery (Shekhar K, et al. (2016) Cell 166(5):1308-1323.e30; and Macosko E Z, et al. (2015) Cell 161(5):1202-1214).

Experimental replicates/sample size: Three individual animals or organoids will be injected for initial FACS analysis. Immunostaining, and other cell counting, will be conducted using a minimum of five sections from at least three individual animals or organoids. MULTI-Seq analysis will be conducted using tissue pooled from a minimum of three individual samples for each construct tested. Functional studies using SLED constructs will be conducted using a minimum of 5 individuals or organoids.

Controls used: The cell counting data will be performed blind (Liu K, et al. (2017) Nature 548(7669):582-587).

Consideration of sex as a biological variable: None of alternative splicing patterns used for design of SLED constructs appear to show any clear sexual dimorphism. To address any unexpected effects of sex on splicing or reporter expression, equal numbers of male and female animals will be used for the studies described herein.

### Example 5: Using Alternative Splicing to Induce Photoreceptor-Specific Gene Expression in the Retina and Neuron Specific Gene Expression in the Brain

Two proof-of-concept plasmids were generated. First, a plasmid that selectively expresses GFP in neurons and a second plasmid that selectively expresses GFP in photoreceptors.

Protocol: To create a sequence that can drive cell type-specific expression, the method can comprise the following steps:
1. Identify an intron that contains an exon that is selectively spliced in the cell type of interest.
2. Extend this intron sequence into the flanking 5′ and 3′ exons, ˜50-100 bp on either end, to ensure that the appropriate exonic splicing enhancer sequences are available.
3. Add a kozak sequence and ATG start codon upstream of the intron cassette that is in-frame with a coding sequence that does not contain premature stop codons in the canonically spliced reading frame.
4. Insert a mutation into the cell type-specific exon that shifts the reading frame. This can be the insertion of N*3+1 basepairs (e.g. +1, +4, +7, etc.), the insertion of N*3+2 basepairs (e.g. +2, +5, +8, etc.), the deletion of N*3-1 basepairs (e.g. −1, −4, −7, etc.), the deletion of N*3-2 basepairs (e.g. −2, −5, −7, etc.) or a combination of the above that leads to an exon length that is not a multiple of 3.
5. Add gene of interest in-frame with the reading frame that results from the splicing-in of the cell type-specific exon.
6. Make point mutations in the sequence to remove any premature stop codons.

Insert this intron cassette into an AAV packaging plasmid for virus preparation.

FIG. 9 shows a neuron-specific expression plasmid and a photoreceptor-expression plasmid along with specific fluorescent markers.

Exons chosen for these experiments were derived from a database generated for this purpose using the following database: http://ascot.cs.jhu.edu/; and https://www.biorxiv.org/content/10.1101/501882v1.

Many other candidate exons exist. For example, 80 exons were identified that are highly-specific to photoreceptors, as well as the intron coordinates that would be used in generating a gene therapy construct. Other tables/coordinates can be generated for various cell types.

The neuronal intron sequence, with the neuron-specific exon bolded and underlined ([C] nucleotide is the +1 insertion mutation added to shift the reading frame):

(SEQ ID NO: 1)
ATGACTAGTAGATGGGCCAACTTCCATCTGGAAAACTCAGGCTGGCAAA
AGATCAACAACTTCAGCGCTGACATCAAGGTAACTGTGCAAAAAACCAT
AGCGTTCAGGTAGATACGATGGCTTCAGGGATTTTATCCCATTGTAACA
GAGACAAAAATGATAGGATATCACCAGGCCCTGTTGTCTTTCCCTAGGA
GCTAATTTCCTTAAGTATATTCTGATTTTAAAAGGTTAAATGCTCTCTT
GCCAACCATATGTGTTTCTCGTTTAAGCTTTTCTGTATTTCAACTTTTG
ATTTAACATACAATTAACATAGTAGATGTTGTTTCCATAGGTTGCTACT
CATCATGCTTTGTAATGCCATAATGTGTTCATTTTCTCTTTCCCTATGC
TTCCTGGATTTCTGCTCTTCTATTTCATGCTTGTTTATCTGTCAAGCTT
ATTGACTTCAGTAA[C]TTCAGTGAAGGTACAGAAATACTTAATAGCTT
TATTGGGTTGATGGCATCGATAAATTGTCAAGTCCTAACAATCACGTGC
AAAGACAAAAAGAAAGATCTCTCAAATAAGAAGACAGAGAAGAATGCCT
TATATTATCACAAAAGCTGCTCCCAATGTCCTCATCACCAAGGGACAAT
AAGATGCAGCTACTCTTTGGCAGTGTGATCACACTATTATGTTCAATTC
TTTTGTAGGATTCCAAAGCCTATTTCCATCTGCTCAATCAAATCGCACC
GAAGGGA

The photoreceptor intron sequence, with photoreceptor-specific exon bolded and underlined ([T] nucleotide is the +1 insertion mutation added to shift the reading frame, {G} nucleotide is a point mutation intended to remove premature stop codon):

(SEQ ID NO: 4)
ATGACTAGGATCAACTTCTATGCAGGGGCAAACCAGAGCATGAATGTCA
CTTGTGTTGGCAAGGTGAGTGTGGGGGCCCTCCTTACCTGCCCACCTGG
TTAGACTTCCTGGTTTCTGAGTGCTTCACCCATATCTCCCTATCTTTTT
GTGCTTTCAGAGGCCACAG[T]CACTA{G}AGGGACAAGGGGGTAAGAG
TGGGCGCCTATGCAGTTTTAGCTCTAAGAGGCTCTTAGCCCTATTGCTT
CTCTCTAGGATAAAGAGAGCCTGCTGTCCTGGAGATAGACCTATCCCTT
CCTGCACCAAAGCTCTGACCTCTGGTTCCTTCCCTGTCAACTTTTTCTT
ACATCTCAGTTGTCTGGGTTTCTTCCACTCTCCCATCATGCCTTGTTTC
TCAGTTCCCTCAGTCTGCTAGCTACTGCTCAGTTAGCACCCTTTGCTAC
AACTAGTTGTCCTTGGAACCCTGCAGCCAACTCTGTCCTCTCTAGAAAC
TCTCCTCCTTCCCACTGAGCCTTGACTGTTTATCTGTTCTTTCTTGGCT
CTGCTCCAGAGACTGATTCCCAAGGACGGGGTAAGAACTTGGGGATTGA
TGGTGGAGTTAGAAGGCCCTCACCGTGTTGTCAGCACCCTTAGAAGACC
TAGTCTGATGGGAGATAGGCCACCCCTATCTGCAGACATGCAGATAGGA
ACATGTGTGCATGCGCACACACAAATGCACACACAGCTACCTGAGCAGA
TGCACAGCTCAAAGAAAACAAGTTTGACAGGGATAATTTGGGATGAAGG
AGGTACAGAAGGAAGTCTTGTGAGCGCTTCCAGGTGCCTGCTGTTCCTA
ACATCCTCTCCCCTTAACCTTCCTGCACCCCCACAGAGAGAGAAGATGC
TGAGAACCTTGGCCACTTTGTCATGTTCCCTGCTAATGGCAGCA.

Photoreceptors, the light sensing cells of the retina, exhibit uncommon splicing events that are not found in any other cell type. Vectors were designed that use these photoreceptor-specific splicing events to deliver nucleic acid constructs to expression a gene in a specific cell type. In cells that are not photoreceptors, normal splicing will generate a sequence that encodes a red fluorescent protein. In photoreceptors, however, a photoreceptor-specific exon is incorporated, leading to a frameshift and the expression of a green fluorescent protein.

Importantly, this method of cell type-specific gene expression is achieved through an entirely promoter-independent mechanism. This allows the use of any promoter to drive high levels of protein expression, while maintaining stringent cell type specificity. Furthermore, the methods and compositions disclosed herein can target cell types for which no cell type-specific promoter exists.

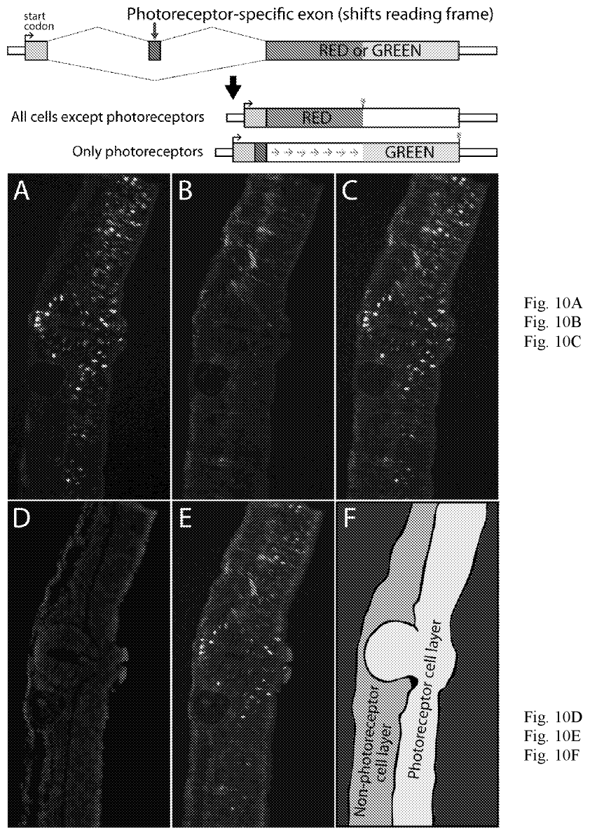

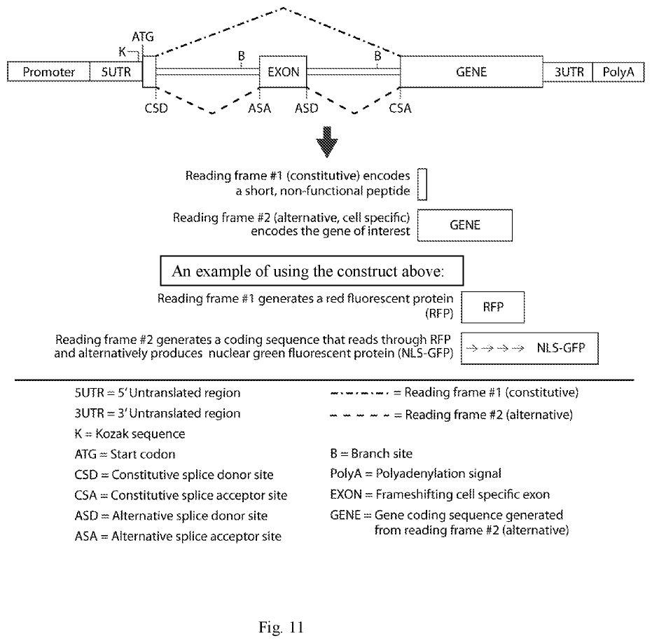

FIG. 10 validates the disclosed method in a proof-of-concept experiment by electroporating mouse retinas with a construct designed using the protocol described herein.

### Example 6: Using Alternative Splicing to Rescue a Disease Phenotype

The compositions and methods disclosed herein can be used to rescue the rd10 mouse model (https://www.jax.org/strain/004297). The rd10 mouse is a point mutation in the gene Pde6b and is used as a model for retinitis pigmentosa. The Pde6b gene will be cloned into a photoreceptor-specific construct, packaged in the AAV2.7m8 serotype, and the virus will be injected into rd10 mice. Photoreceptor survival and function will be assayed to determine efficacy.

### Example 7: Using Alternative Splicing In Vitro to Selectively Kill Cancer Cells

The methods and compositions described herein can be used to specifically target cancer cells that exhibit cancer-specific splicing events. The SF3B1 gene is commonly mutated in many forms of cancers. SF3B1 mutations result in the splicing of unique alternative exons that are only found in SF3B1-mutated cancers. In FIG. 12, an SF3B1-linked exon is cloned into a bichromatic construct to demonstrate selective delivery of GFP to only SF3B1-mutated cancer cells. FIG. 12A shows RTPCR analysis across three uveal melanoma cell lines (92.1, OMM1, Mel202) in which only Mel202 has an SF3B1 mutation. An arrow indicates the incorporation of a novel exon in only the Mel202 cell line. FIG. 12B is a schematic of an SF3B1 mutation-specific expression plasmid. FIG. 12C shows that mCherry is expressed in all three cell lines while GFP is only expressed in the Mel202 cell line. To selectively kill SF3B1-mutated cancer cells, GFP is replaced with a protein that can induce cell death or an immunogenic response.

### Example 8: Using Alternative Splicing to Rescue the Rd2 Mouse Model

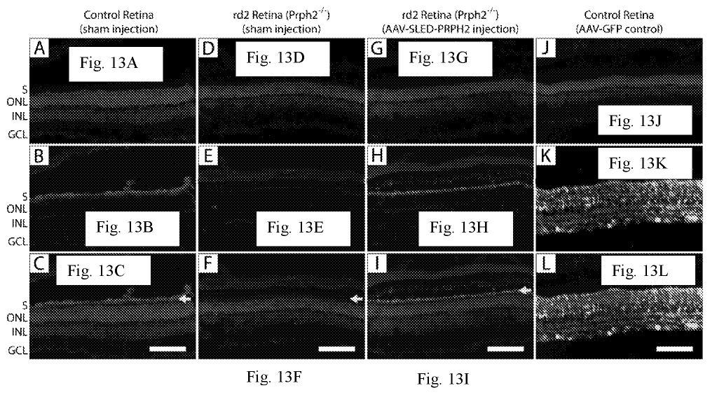

The rd2 mouse (https://www.jax.org/strain/001981) is a mouse that contains a point mutation in the gene Prph2 and is used as a model for retinitis pigmentosa. FIG. 13 shows an experiment where alternative splicing is used to rescue the rd2 mouse model. Image panels indicate DAPI nuclear stain (FIGS. 13A, D, G, J), immunohistochemical staining of Prph2 (FIGS. 13B, E, H), GFP fluorescence (FIG. 13K), and composite overlays (FIGS. 13C, F, I, L). Sham injected control retinas (FIGS. 13A, B, C) show the presence of photoreceptor segments as indicated by Prph2 labeling (arrow in FIG. 13C). In sham injected rd2 mouse retinas (FIGS. 13D, E, F), photoreceptor segments are dysfunctional due to loss of Prph2 expression (arrow in FIG. 13F). When the Prph2 gene is cloned into a photoreceptor-specific construct with a ubiquitous promoter, packaged in the AAV2-7m8 serotype, and injected into rd2 mice (FIGS. 13G, H, I), photoreceptor segments are recovered due to restoration of normal Prph2 expression patterns (arrow in FIG. 131). Control retinas injected with AAV2-7m8 virus expressing GFP with a ubiquitous promoter (FIG. 13J, K, L) show broad infectivity and GFP expression across the layers of the retina. However, the photoreceptor-specific construct (FIGS. 13G, H, I) can restrict Prph2 expression to the photoreceptor layer by using alternative splicing. (Abbreviations: S=Segments, ONL=Outer Nuclear Layer, INL=Inner Nuclear Layer, GCL=Layer of Ganglion cells)

What is claimed is:

1. A nucleic acid construct comprising:
a) a start codon; and
b) an intron cassette, wherein the intron cassette comprises a cell specific exon sequence, a splice donor site, a branch site, and an acceptor site, wherein the cell specific exon sequence is out of frame with the start codon and comprises one or more frameshift mutations.

2. The nucleic acid construct of claim 1, wherein the splice donor site is upstream from the cell specific exon sequence within the intron cassette.

3. The nucleic acid construct of claim 1, wherein the splice acceptor site is downstream from the cell specific exon sequence within the intron cassette.

4. The nucleic acid construct of claim 1, wherein the branch site is upstream or downstream from the cell specific exon sequence within the intron cassette.

5. The nucleic acid construct of claim 1, wherein the start codon is upstream from the intron cassette.

6. The nucleic acid construct of claim 1, wherein the cell specific exon sequence is flanked by sequences of the intron cassette.

7. The nucleic acid construct of claim 1, wherein the cell specific exon sequence is specific for a cell type, wherein the cell type is a neuron, a skeletal muscle cell, a cochlear hair cell, an oligodendrocyte or a photoreceptor cell.

8. The nucleic acid construct of claim 7, wherein the cell specific exon sequence is spliced in-frame to the start codon upon introducing the nucleic acid construct to the specific cell type.

9. The nucleic acid construct of claim 1, further comprising a Kozak sequence.

10. The nucleic acid construct of claim 9, wherein the Kozak sequence is upstream of the intron cassette.

11. The nucleic acid construct of claim 10, wherein the Kozak sequence is upstream of the intron cassette and is out of frame with the cell specific exon sequence.

12. The nucleic acid construct of claim 1, wherein the cell specific exon sequence does not comprise a premature stop codon in a canonically spliced reading frame.

13. The nucleic acid construct of any of claims 1-12, further comprising a promoter.

14. The nucleic acid construct of claim 1, further comprising a 5′ untranslated region (5′UTR).

15. The nucleic acid construct of claim 14, wherein the 5′UTR is positioned between a promoter and the start codon.

16. The nucleic acid construct of claim 13, wherein the Kozak sequence is operatively linked to the promoter, wherein the promoter is upstream of the Kozak sequence.

17. The nucleic acid construct of claim 13, wherein the promoter is regulatable.

18. The nucleic acid construct of claim 13, wherein the promoter is constitutively active.

19. The nucleic acid construct of claim 1, further comprising a gene of interest.

20. The nucleic acid construct of claim 19, wherein the gene of interest is downstream of the intron cassette.

21. The nucleic acid construct of claim 19, wherein the gene of interest is in-frame with the reading frame after the cell specific exon is spliced in.

22. The nucleic acid construct of claim 19, wherein the gene of interest is a therapeutic agent or a detectable moiety.

23. The nucleic acid construct of claim 1, further comprising a polyadenylation signal.

24. The nucleic acid construct of claim 1, further comprising a 3′ untranslated region (3′UTR).

25. The nucleic acid construct of claim 24, wherein the 3′UTR is positioned between a gene of interest and a polyadenylation signal.

26. A cell comprising any of the nucleic acid constructs of claims 1-25.

27. A vector comprising any of the nucleic acid constructs of claims 1-25.

28. The vector of claim 27, further comprising a selectable marker.

29. A method of expressing a nucleotide sequence in a specific cell, the method comprising introducing the nucleic acid construct of any of claims 1-25 to the specific cell.

30. The method of claim 29, wherein the specific cell is a eukaryotic cell.

31. The method of claim 30, wherein the eukaryotic cell is a mammalian cell.

32. The method of claim 30, wherein the mammalian cell is a neuron, a skeletal muscle cell, a cochlear hair cell, an oligodendrocyte or a photoreceptor cell.

33. The method of claim 32, wherein the mammalian cell is a diseased cell.

34. A method of treating a human patient, the method comprising: administering the nucleic acid construct of any of claims 1-25 to the human patient.

35. The method of claim 34, wherein the human patient has been identified as being in need of treatment.

36. The method of claim 34, wherein the human patient has a disease.

37. The method of claim 36, wherein the disease is monogenetic disease.

38. A method of delivering a therapeutic agent to one or more cells, the method comprising: contacting the one or more cells with the nucleic acid construct of any of claims 19-22.

39. The method of claim 38, wherein the one or more cells is a neuron, a skeletal muscle cell, a cochlear hair cell, an oligodendrocyte or a photoreceptor cell.

40. A method of selectively inducing exon splicing in a cell, the method comprising contacting a cell with the nucleic acid construct of any of claim 1-25.

41. A nucleic acid construct comprising:
a) a first intron sequence comprising a constitutive splice donor site, a branch site, and an alternative splice acceptor site;
b) a cell specific exon sequence; and
c) a second intron sequence comprising an alternative splice donor site, a branch site, and an constitutive splice acceptor site.

42. A nucleic acid construct comprising from 5′ to 3′: a first intron sequence comprising a constitutive splice donor site, a branch site, and an alternative splice acceptor site; a cell specific exon sequence; and a second intron sequence comprising an alternative splice donor site, a branch site, and an constitutive splice acceptor site.

43. The nucleic acid construct of claim 41 or 42, further comprising a start codon, wherein the start codon is upstream from the first intron sequence.

44. The nucleic acid construct of claim 41 or 42, wherein cell specific exon sequence is flanked by the first intron sequence and the second intron sequence.

45. The nucleic acid construct of claim 41 or 42, wherein the cell specific exon sequence is specific for a cell type, wherein the cell type is a neuron, a skeletal muscle cell, a cochlear hair cell, an oligodendrocyte or a photoreceptor cell.

46. The nucleic acid construct of claim 45, wherein the cell specific exon sequence is spliced in-frame to the start codon upon introducing the nucleic acid construct to the specific cell type.

47. The nucleic acid construct of claim 41 or 42, further comprising a Kozak sequence.

48. The nucleic acid construct of claim 47, wherein the Kozak sequence is upstream of the first intron sequence.

49. The nucleic acid construct of claim 48, wherein the Kozak sequence is upstream of the first intron sequence and is out of frame with the cell specific exon sequence.

50. The nucleic acid construct of claim 41 or 42, wherein the cell specific exon sequence does not comprise a premature stop codon in a canonically spliced reading frame.

51. The nucleic acid construct of any of claims 41-50, further comprising a promoter.

52. The nucleic acid construct of claim 41 or 42, further comprising a 5′ untranslated region (5′UTR).

53. The nucleic acid construct of claim 52, wherein the 5′UTR is positioned between a promoter and the start codon.

54. The nucleic acid construct of claim 51, wherein the Kozak sequence is operatively linked to the promoter, wherein the promoter is upstream of the Kozak sequence.

55. The nucleic acid construct of claim 51, wherein the promoter is regulatable.

56. The nucleic acid construct of claim 51, wherein the promoter is constitutively active.

57. The nucleic acid construct of claim 41 or 42, further comprising a gene of interest.

58. The nucleic acid construct of claim 57, wherein the gene of interest is downstream of the second intron sequence.

59. The nucleic acid construct of claim 57, wherein the gene of interest is in-frame with the reading frame after the cell specific exon is spliced in.

60. The nucleic acid construct of claim 57, wherein the gene of interest is a therapeutic agent or a detectable moiety.

61. The nucleic acid construct of claim 41 or 42, further comprising a polyadenylation signal.

62. The nucleic acid construct of claim 41 or 42, further comprising a 3′ untranslated region (3′UTR).

63. The nucleic acid construct of claim 62, wherein the 3′UTR is positioned between a gene of interest and a polyadenylation signal.
